(19) United States
(12) Patent Application Publication
(10) Pub. No.: US 2011/0318830 A1
(43) Pub. Date: Dec. 29, 2011

(54) COMPOSITIONS AND METHODS FOR RE-PROGRAMMING AND RE-DIFFERENTIATING CELLS

(75) Inventors: Steven P. Briggs, Del Mar, CA (US); Kiyoshi Tackihawa, San Diego, CA (US); Robert O'brien, La Jolla, CA (US)

(73) Assignee: THE REGENTS OF THE UNIVERSITY OF CALIFORNIA, Oakland, CA (US)

(21) Appl. No.: 13/126,703

(22) PCT Filed: Nov. 12, 2009
(86) PCT No.: PCT/US2009/064132

§ 371 Date: Aug. 18, 2011

Related U.S. Application Data
(60) Provisional application No. 61/113,939, filed on Nov. 12, 2008.

(51) Int. Cl. C12N 5/071; C07K 14/00; C07H 21/04

(52) U.S. Cl. 435/350; 435/377; 435/366; 435/363; 435/354; 435/353; 435/352; 435/351; 536/23.1; 530/350

(57) ABSTRACT

The invention provides compositions and in vitro and ex vivo methods for de-differentiating or re-programming mammalian cells. In alternative embodiments, the invention provides compositions comprising mixtures of Designed Regulatory Proteins (DRPs) or Reprogramming DRP protein (ReD) for de-differentiating or re-programming mammalian cells. The invention also provides compositions and methods for direct reprogramming of a first differentiated phenotype of a cell to a second differentiated phenotype.

### RELATED APPLICATIONS

This patent application has as a priority document (claims the benefit of priority of) U.S. Provisional Application No. 61/113,939, filed Nov. 12, 2008. The aforementioned application is expressly—explicitly incorporated herein by reference in its entirety and for all purposes.

### TECHNICAL FIELD

This invention relates to cellular and developmental biology and regenerative medicine. The invention provides compositions and in vitro and ex vivo methods for de-differentiating or re-programming mammalian cells. In alternative embodiments, the invention provides compositions comprising mixtures of Designed Regulatory Proteins (DRPs) for de-differentiating or re-programming mammalian cells. The invention also provides compositions and methods for direct reprogramming of a first differentiated phenotype of a cell to a second differentiated phenotype.

### BACKGROUND

Current protocols for cell programming use DNA transduction to introduce programming genes. This causes permanent genetic modifications that can perturb cell fate in unpredictable ways, such as causing cancer.

Reprogramming human fibroblasts to become induced pluripotent stem (iPS) cells has opened the door to the ethical derivation of stem cell lines with clinical and research potential. Takahashi (2006) Cell 126(4):663-676; Takahashi (2007) Cell 131(5):861-872; and Okita (2007) Nature 448(7151):313-317, introduced four transgenes: Oct4, Sox2, Klf4 and c-Myc, into both mouse and human fibroblasts and recovered colonies at a ratio of 1/5,000 cells that displayed molecular, phenotypic, and developmental properties that are characteristic of embryonic stem cells (ES cells). Yu (2007) Science 318(5858):1917-1920, obtained colonies at a ratio of 1/10,000 cells using a slightly different gene cocktail (Oct4, Sox2, Nanog, and Lin28). In these protocols, derivation of iPS cells requires genetic modification of the cell; this can cause mutations leading to cancer or other illness. These lines are not safe for clinical use and disease models derived from them—though useful in the short term—may bear artifacts.

### SUMMARY

In alternative embodiments, the invention provides compositions and methods comprising or using mixtures of Designed Regulatory Proteins (DRPs) or Reprogramming DRP proteins (ReDs) for de-differentiating or re-programming mammalian cells; and alternatively for direct reprogramming of a first differentiated phenotype of a cell to a second differentiated phenotype.

The invention provides compositions comprising a plurality of Designed Regulatory Proteins (DRPs) or Reprogramming DRP proteins (ReDs), or provides a plurality of DRPs or ReDs, or the composition comprises one or at least one DRP or ReD chimeric protein that can bind to and activate the transcription of each member of the combination of genes set forth below, wherein
(a) each DRP or ReD is a chimeric protein comprising:
   (1) at least one zinc finger DNA binding peptide domain specific for (capable of specifically binding to) a promoter or a transcriptional regulatory region of a gene;
   (2) at least one nuclear localization peptide (NLP) domain;
   (3) at least one cell-penetrating peptide (CPP); and,
   (4) a transcription activation peptide domain and/or a transcription repression peptide domain; and
(b) at least one transcription activation peptide domain of each DRP or ReD chimeric protein can bind to and activate the transcription of at least one of the following genes, and the composition comprises at least one DRP or ReD chimeric protein that can bind to and activate the transcription of each member of the combination of genes selected from the group consisting of:

(1) a combination of genes consisting of an Oct4, a Sox2, a Klf4, a c-Myc, a Lin28 and a Nanog gene;

(2) a combination of genes consisting of any five of the following genes: Oct4, Sox2, Klf4, c-Myc, Lin28, Nanog;

for example, a combination of genes consisting of a Sox2, a Klf4, a c-Myc, a Lin28 and a Nanog gene; an Oct4, a Klf4, a c-Myc, a Lin28 and a Nanog gene; an Oct4, a Sox2, a c-Myc, a Lin28 and a Nanog gene; an Oct4, a Sox2, a Klf4, a Lin28 and a Nanog gene; an Oct4, a Sox2, a Klf4, a c-Myc and a Nanog gene; or, an Oct4, a Sox2, a Klf4, a c-Myc and a Lin28 gene;

(3) a combination of genes consisting of any four of the following genes: Oct4, Sox2, Klf4, c-Myc, Lin28, Nanog;

for example, a combination of genes consisting of Oct4, Sox2, Klf4, c-Myc; or, Sox2, Klf4, c-Myc, Lin28; or, Klf4, c-Myc, Lin28, Nanog; etc.

(4) a combination of genes consisting of any three of the following genes: Oct4, Sox2, Klf4, c-Myc, Lin28, Nanog;

for example, a combination of genes consisting of Oct4, Sox2, Klf4; or, Sox2, Klf4, c-Myc; or, c-Myc, Lin28, Nanog; or, Oct4, Sox2, c-Myc; etc.

(5) a combination of genes consisting of any two of the following genes: Oct4, Sox2, Klf4, c-Myc, Lin28; Nanog;

for example, a combination of genes consisting of Oct4, Sox2; or, Klf4, c-Myc; or, Lin28; Nanog; or, Oct4, c-Myc; etc.

(6) a combination of genes consisting of an Oct4 or a Sox2 gene, and a Klf4 or a Nanog gene;

(7) a combination of genes consisting of an Oct4, a Sox2, and a Klf4 or a Nanog gene; or

(8) a combination of genes consisting of an Oct4 gene, or a Sox2 gene, or a Klf4 or a Nanog gene.

In one embodiment, the at least one DRP chimeric protein comprises a recombinant protein, a synthetic protein, a peptidomimetic, a non-natural peptide, or a combination thereof.

In alternative embodiments, the chimeric protein comprises multiple copies of the zinc finger DNA binding peptide domain, the NLP, the CPP and/or the transcription activation peptide.

In alternative embodiments, a different DRP or ReD chimeric protein binds to and activates the transcription of each gene in the combination, or one of the DRP chimeric proteins can bind to and activate the transcription of two different genes in the combination, or one of the DRP or ReD chimeric proteins can bind to and activate the transcription of three or more different genes in the combination.

In alternative embodiments, the combination of genes further comprises at least one member of the Myc family of transcription factors; or the at least one member of the Myc family of transcription factors is a N-Myc, a L-Myc or a c-Myc gene.

In alternative embodiments, the least one DRP or ReD chimeric protein has or further comprises at least one transcription repression peptide domain that represses the transcription of a Pax5 message (mRNA, transcript).

In alternative embodiments, the at least one DRP or ReD chimeric protein has or further comprises at least one transcription repression peptide domain that represses the transcription of a (zinc finger transcription factor) GATA6 gene, or the repression peptide domain comprises a Krüppel-associated box (KRAB) domain of KOX1, or the repression peptide domain comprises an SRDX domain from Arabidopsis thaliana SUPERMAN protein.

In alternative embodiments, the at least one zinc finger binding peptide domain comprises (1) a zinc-finger of the C₂H₂ class; (2) a zinc-finger of the C₄ class; or (3) a zinc-finger of C₆ class; or the at least one zinc finger binding peptide domain comprises the consensus sequence Cys-X₂₋₄-Cys-X₃-Phe-X₅-Leu-X₂-His-X3-His (SEQ ID NO:1).

In alternative embodiments, the at least one nuclear localization peptide (NLP) domain comprises: (1) an NLP sequence of a large T antigen of the simian virus 40 (SV-40), or PKKKRKV (SEQ ID NO:2); (2) a consensus sequence fitting B₄ (SEQ ID NO:3), P(B₃X) (SEQ ID NO:4), PXX(B₃X) (SEQ ID NO:5), B₃(H/P) (SEQ ID NO:6), where B is a basic amino acid, P is proline, H is histidine, X is any amino acid and letters in parentheses can be in any order; (3) a bipartite NLP comprising two short stretches of basic amino acids separated by a non-conserved sequence; or, (4) a cellular nucleoplasmin protein KRPAATKKAGQAKKKK (SEQ ID NO:7).

In alternative embodiments, wherein the at least one cell-penetrating peptide (CPP) comprises: (1) a plurality of polycationic amino acid residues; (2) a plurality of arginine amino acid residues; or (3) a TAT protein (Trans-acting Activator of Transcription) of a Human Immunodeficiency Virus (HIV-1).

In alternative embodiments, of the composition of the invention: (1) the at least one transcription activation peptide domain is at least approximately 25% hydrophobic and is linked to the at least one zinc finger binding peptide in a manner that does not interfere with the promoter or a transcriptional regulatory binding activity of the zinc finger DNA binding peptide, and the transcription activation peptide is both necessary and sufficient to activate transcription of the gene; and/or, (2) the transcription activation peptide domain is between about 5 to 25 amino acids in length, or is between about 6 to 20 amino acids in length, or is about 5, 6, 7, 8, 9, 10, 11, 11, 12, 13, 14 or 15 amino acids in length.

In alternative embodiments, the at least one transcription activation peptide domain comprises a herpes simplex virus (HSV) VP-16 activation peptide domain or a peptide derived from the C-terminal transcription activation domain of β-catenin (FDTDL).

In alternative embodiments, at least one, or all, of the DRP or ReD chimeric proteins further comprises, or is attached to, a lipid or a polyethylene glycol (PEG) moiety; or, at least one, or all, of the DRP or RED chimeric proteins further comprises, or is attached to, an epitope peptide tag or a detectable composition or moiety.

In alternative embodiments, the composition comprises a phosphoprotein, a fluorescent molecule, a fluorescent tagged protein, a radiolabel or a radiolabeled protein.

In alternative embodiments, the composition further comprises a small molecule, a hormone or a cytokine that has a de-differentiation (re-programming) effect on the mammalian cell; in one aspect, the cytokine comprises a transforming growth factor-beta (TGF-beta).

In alternative embodiments, the composition further comprises a large T antigen of the simian virus 40 (SV-40), or any protein or peptide that inhibits the activity of tumor suppressor gene retinoblastoma-1 (RB1) and/or p53 tumor suppressor gene (TP53).

In alternative embodiments, the composition further comprises a protein or peptide comprising or consisting of a catalytic subunit of TERT, e.g., in one aspect the catalytic subunit of TERT is hTERT.

In alternative embodiments, the composition further comprises a histone deacetylase inhibitor, or the histone deacetylase inhibitor comprises a valproic acid (VPA).

In alternative embodiments, a Designed Regulatory Protein (DRP) or a Reprogramming DRP protein (ReD) used to practice this invention: is encoded by a nucleic acid comprising SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, or SEQ ID NO:30; or, has an amino acid sequence comprising SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.

The invention provides liquids, gels, hydrogels, powders and/or aqueous formulations comprising at least one composition of the invention.

The invention provides vesicles, liposomes, nanoparticles or nanolipid particles (NLPs) comprising at least one composition of the invention, and/or the liquid, gel, hydrogel, powder or aqueous formulation of the invention.

The invention provides isolated or cultured cells comprising (or having contained therein) at least one composition of the invention, and/or the liquid, gel, hydrogel, powder or aqueous formulation of the invention, and/or the vesicle, liposome, nanoparticle or nanolipid particle (NLP) the invention. In alternative aspects, the cell is a mammalian cell, or the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell. The invention provides pharmaceuticals or sterile formulations comprising the mammalian cell of the invention. The invention provides products of manufacture comprising an isolated or cultured cell of the invention. The invention provides artificial organs or implants comprising an isolated or cultured cell of the invention. In alternative aspects, the artificial organs or implants of the invention (e.g., comprising an isolated or cultured cell of the invention) comprise or form an artificial tissue or organ, or an orthopedic implant, an ocular implant, a dental implant, an auricular implant, or a heart valve bio-prosthesis, or a bioactive wound dressing.

The invention provides in vitro or ex vivo methods for de-differentiating or re-programming a mammalian cell comprising:
(a) (i) providing
   (1) at least one composition of the invention,
   (2) the liquid or aqueous formulation of the invention,
   (3) the vesicle, liposome, nanoparticle or nanolipid particle of the invention, or
   (4) a plurality of Designed Regulatory Proteins (DRPs) or Reprogramming DRPS (ReDs), wherein each DRP or ReD is a chimeric protein comprising: (I) at least one zinc finger DNA binding peptide domain specific for (capable of specifically binding to) a promoter or a transcriptional regulatory region of a gene, (II) at least one nuclear localization peptide (NLP) domain, (III) at least one cell-penetrating peptide (CPP), and (IV) a transcription activation peptide domain and/or a transcription repression peptide domain;
   wherein the at least one transcription activation peptide domain of each DRP chimeric protein can bind to and activate the transcription of at least one (or more) of the following genes, and the plurality comprises at least one DRP or ReD chimeric protein that can bind to and activate the transcription of each member the combination of genes selected from the group consisting of:
      (1) a combination of genes consisting of an Oct4, a Sox2, a Klf4, a c-Myc, a Lin28 and a Nanog gene;
      (2) a combination of genes consisting of a Sox2, a Klf4, a c-Myc, a Lin28 and a Nanog gene;
      (3) a combination of genes consisting of an Oct4, a Klf4, a c-Myc, a Lin28 and a Nanog gene;
      (4) a combination of genes consisting of an Oct4, a Sox2, a c-Myc, a Lin28 and a Nanog gene;
      (5) a combination of genes consisting of an Oct4, a Sox2, a Klf4, a Lin28 and a Nanog gene;
      (6) a combination of genes consisting of an Oct4, a Sox2, a Klf4, a c-Myc and a Nanog gene;
      (7) a combination of genes consisting of an Oct4, a Sox2, a Klf4, a c-Myc and a Lin28 gene;
      (8) a combination of genes consisting of any four of the following genes: Oct4, Sox2, Klf4, c-Myc, Lin28; Nanog;
      (9) a combination of genes consisting of any three of the following genes: Oct4, Sox2, Klf4, c-Myc, Lin28; Nanog;
      (10) a combination of genes consisting of any two of the following genes: Oct4, Sox2, Klf4, c-Myc, Lin28; Nanog;
      (11) a combination of genes consisting of an Oct4 or a Sox2 gene, and a Klf4 or a Nanog gene
      (12) a combination of genes consisting of an Oct4, a Sox2, and a Klf4 or a Nanog gene;
      (13) a combination of genes consisting of an Oct4 gene, or a Sox2 gene, or a Klf4 or a Nanog gene; or
      (14) the combination of genes of any of (1) to (13), wherein the combination of genes further comprises at least one member of the Myc family of transcription factors, or an N-Myc, L-Myc or c-Myc gene;
(ii) providing a mammalian cell more differentiated than a pluripotent phenotype; and
(iii) contacting in vitro or ex vivo the composition, or the liquid or aqueous formulation, or the vesicle, liposome, nanoparticle or nanolipid particle, or the plurality of DRPs, with the mammalian cell in an amount effective to cause the de-differentiation or re-programming of the mammalian cell.

In alternative embodiments of the methods of the invention, the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

In alternative embodiments of the methods of the invention, the in vitro or ex vivo contacting of the mammalian cell with the composition, or the liquid or aqueous formulation, or the vesicle, liposome, nanoparticle or nanolipid particle, or the plurality of DRPs or ReDs, is in an aqueous cell culture environment, or the in vitro or ex vivo contacting is on mammalian cells embedded in a gel, or the in vitro or ex vivo contacting is on a mammalian cell that is adherent on (to) a plate or a fixed or gel structure.

In alternative embodiments of the methods of the invention, the mammalian cell is contacted with the composition, or the liquid or aqueous formulation, or the vesicle, liposome, nanoparticle or nanolipid particle, or the plurality of DRPs or ReDs, in an amount effective to cause the de-differentiation or re-programming of the mammalian cell to a pluripotent cell.

In alternative embodiments of the methods of the invention, the mammalian cell is contacted with the composition, or the liquid or aqueous formulation, or the vesicle, liposome, nanoparticle or nanolipid particle, or the plurality of DRPs or ReDs, in an amount effective to cause the de-differentiation or re-programming of the mammalian cell to a pluripotent cell, a multipotent stem cell, a unipotent stem cell or a totipotent stem cell.

In alternative embodiments of the methods of the invention, the mammalian cell of step (a)(ii), before de-differentiation or re-programming, is an endodermal cell, a mesodermal cell or an ectodermal cell.

In alternative embodiments of the methods of the invention, the mammalian cell of step (a)(ii), before de-differentiation or re-programming, is an adult stem cell, an embryonic stem cell, a somatic stem cell, an adipose-derived stem cell (ASC), a stem cell derived from an epithelial cell or tissue, a hematopoietic stem cell, a mammary stem cell, a mesenchymal stem cell, a neural stem cell, an olfactory adult stem cell, a spermatogonial progenitor cell, a dental pulp-derived stem cell, or a cancer stem cell.

In alternative embodiments of the methods of the invention, the mammalian cell of step (a)(ii), before de-differentiation or re-programming, is an adult somatic cell or an adult germ cell; e.g., in some aspects, the adult somatic cell, before de-differentiation or re-programming, is a hematopoietic cell, a lymphocyte, a macrophage, a T cell, a B cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an embryoid body cell, an ovary cell, a testis cell, an adipose tissue (fat) cell, or a cancer cell.

In alternative embodiments of the methods of the invention, each chimeric protein in the cell culture aqueous environment has a concentration of at least between about 5 to 1000 μgm per ml, or between about 10 to 500 μgm per ml, or between about 50 to 100 μgm per ml; or the mammalian cells are contacted with an aqueous solution or culture media wherein each chimeric protein has a concentration in the aqueous solution or culture media of at least between about 5 to 1000 μgm per ml, or between about 10 to 500 μgm per ml, or between about 50 to 100 μgm per ml; or, 10, 20, 30, 40, 50, 60, 70, 80, 90 or 100 or more μgm per ml.

In alternative embodiments of the methods of the invention, the mammalian cell is cultured for between about one to 24 hours, or between about one to two days; or, the mammalian cell is cultured for between about one to 10 days after the in vitro or ex vivo contacting of step (iii); or, the mammalian cell is cultured before, during and/or after the in vitro or ex vivo contacting of step (iii).

In alternative embodiments of the methods of the invention, the mammalian cell is also contacted with a cytokine that has a de-differentiation (re-programming) effect on the mammalian cell; and in some aspects, the cytokine comprises a transforming growth factor-beta (TGF-beta), interleukin-18 (IL-18, or interferon-γ-inducing factor), adipose complement-related protein or interferon-γ.

In alternative embodiments of the methods of the invention, the mammalian cell is also contacted with a large T antigen of the simian virus 40 (SV-40), or any protein or peptide or nucleic acid that inhibits the activity of a tumor suppressor gene retinoblastoma-1 (RB1) and/or a p53 tumor suppressor gene (TP53), and the contacting is before, during or after the contacting step of (a)(iii).

In alternative embodiments of the methods of the invention, the mammalian cell is also contacted with a protein or peptide comprising or consisting of a catalytic subunit of TERT, or nucleic acid that encodes a catalytic subunit of TERT, and the contacting is before, during or after the contacting step of (a)(iii). The catalytic subunit of TERT can be hTERT.

In alternative embodiments of the methods of the invention, the method further comprises the deletion or inhibition of a gene and/or transcript (mRNA, message) encoding one or more of a set of nucleic acid and/or protein transcription factors responsible for maintaining a differentiated phenotype of the mammalian cell, and/or inhibition of a protein transcription factor responsible for maintaining a differentiated phenotype of the mammalian cell. The deletion or inhibition of a gene and/or transcript (mRNA, message) can be by expression of or administration of a nucleic acid or protein that is inhibitory to the activity and/or expression of the gene, transcript and/or protein transcription factor. In alternative embodiments, the nucleic acid that is inhibitory to the gene and/or transcript comprises an miRNA, an siRNA, a ribozyme and/or an antisense nucleic acid, or the protein that is inhibitory to the activity and/or expression of the gene, transcript and/or protein transcription factor comprises an antibody that specifically binds to the protein transcription factor. In alternative embodiments, the one or more of the transcription factors inhibited is Pax5, or the method further comprises inhibiting or knocking out the expression of a gene and/or transcript encoding Pax5.

In alternative embodiments of the methods of the invention, the method further comprises addition before, during or after the contacting step of (a)(iii) of a histone deacetylase inhibitor. The histone deacetylase inhibitor can comprise a valproic acid (VPA) or related, equivalent compounds.

In alternative embodiments the methods of the invention further comprise identifying and/or isolating the de-differentiated or re-programmed cell by using an antibody that specifically binds to a polypeptide cell surface marker present in the de-differentiated or re-programmed cell and not the cell before de-differentiating or re-programming. In alternative embodiments, the polypeptide cell surface marker present in the de-differentiated or re-programmed cell and not the cell before de-differentiating or re-programming is
(1) CXCR4, CD10, CD13, CD41a (gpIIbIIIa), CD34, CD56, CD90, CD110, CD117, CD123, CD133, CD135, CD277 and/or CD318;
(2) CD10, CD13, CD56, and MHC Class-I cell surface antigens;
(3) the method of (b)(1) or (b)(2), wherein the cells are also negative for
(1) CD3, CD5, CD7, CD11b, CD14, CD15, CD16, CD19, CD25, CD45, and/or CD65 markers, or
(2) CD3, CD4, CD8, CD11c, CD33, CD36, CD38, CD45, CD117, Glycophorin-A and/or HLA DR-II.

In alternative embodiments of the methods of the invention, the cell is identified and/or isolated by positive or negative selection using the antibody. The identifying and/or isolating of the de-differentiated (re-programmed) cell can be by negative selection of cells still expressing a differentiated cell marker. The cell can be identified and/or isolated by fluorescent activated cell sorting (FACS) or affinity column chromatography. The cell can be identified and/or isolated by identification and/or isolation of plasma membrane proteins by mass spectography or chromatography. In one aspect, the identifying and/or isolating the de-differentiated (re-programmed) cell is by determining the presence or absence of a message (mRNA, transcript) determinative of an undifferentiated cell phenotype. The message (mRNA, transcript) determinative of an undifferentiated cell phenotype can be a message for Oct4, a Sox2, a Klf4, a c-Myc, a Lin28 and a Nanog gene.

In alternative embodiments the methods of the invention further comprise implanting the de-differentiated or re-programmed mammalian cell in a vessel, tissue or organ. The de-differentiated or re-programmed mammalian cell can be implanted in the vessel, tissue or organ ex vivo or in vivo. The method also can further comprise implanting the de-differentiated or re-programmed mammalian cell in an individual in need thereof.

The invention provides de-differentiated and/or re-programmed cells made by practicing any method of the invention, wherein the de-differentiated or re-programmed cell is a mammalian cell. The mammalian cell can be a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

The invention provides kits comprising (i) (a) the composition comprising the plurality of Designed Regulatory Proteins (DRPs) or ReDs of the invention, (b) the liquid or aqueous formulation of the invention, or (c) the vesicle, liposome, nanoparticle or nanolipid particle of the invention, or (ii) the kit of (i) further comprising instructions for practicing the methods of any of the invention.

The invention provides in vitro or ex vivo methods for direct reprogramming of a first differentiated phenotype of a cell to a second differentiated phenotype, comprising:

(i) providing a differentiated cell having a first differentiated phenotype;

(ii) identifying a set of transcription factors responsible for maintaining the differentiated phenotype of the differentiated cell;

(iii) identifying a set of nucleic acid and/or protein transcription factors responsible for maintaining the second differentiated phenotype; and

(iv) simultaneously
(1) inhibiting the expression of one or more or all of the nucleic acid and/or protein transcription factors of (a)(ii), wherein by inhibiting the expression of the transcription factors the cell is unable to maintain the first differentiated phenotype; and
(2) activating the expression of the set of transcription factors of (a)(iii), wherein by activating the expression of the set of transcription factors the cell differentiates into the second differentiated phenotype,

thereby directly reprogramming the cell from a first differentiated phenotype to a second differentiated phenotype.

In alternative embodiments of this method, the direct reprogramming step further comprises, or also comprises, contacting the cell with the composition of the invention, or the liquid, gel, hydrogel, powder or aqueous formulation of the invention, or the vesicle, liposome, nanoparticle or nanolipid particle (NLP) of the invention.

In alternative embodiments, the expression of one or more or all of the transcription factors of (a)(ii) are by inhibited by deletion or knocking out of a gene encoding one or more of the set of transcription factors responsible for maintaining the first differentiated phenotype. The expression of one or more or all of the transcription factor(s) of (a)(ii) can be inhibited by deletion or inhibition of a transcript (mRNA, message) encoding one or more of a set of protein transcription factors responsible for maintaining the first differentiated phenotype, and/or the activity of one or more or all of the transcription factor(s) of (a)(ii) are inhibited by direct inhibition of the activity of one or more or all protein transcription factor(s) responsible for maintaining the first differentiated phenotype. The deletion or inhibition of a transcript (mRNA, message) encoding one or more of a set of protein transcription factors can be by expression of or administration of a nucleic acid or protein that is inhibitory to the one or more of the set of protein transcription factors, or an antibody directly inhibits the activity of one or more or all protein transcription factor(s) responsible for maintaining the first differentiated phenotype. The nucleic acid that is inhibitory to the one or more of the set of protein transcription factors can comprise an miRNA, an siRNA, a ribozyme and/or an antisense nucleic acid. In alternative embodiments, one of the transcription factors inhibited is Pax5, or the method of (a)(iv)(1), further comprising inhibiting or knocking out the expression of Pax5.

In alternative embodiments the method further comprises addition of a histone deacetylase inhibitor, e.g., wherein the histone deacetylase inhibitor comprises a valproic acid (VPA).

In alternative embodiments the method further comprises expressing or upregulating a methyltransferase gene or enzyme to maintain the second differentiated phenotype. The first differentiated phenotype can be a keratinocyte that is reprogrammed to a second differentiated phenotype selected from the group consisting of a nerve cell or an astrocyte.

In alternative embodiments the method further comprises implanting the re-programmed differentiated cell in a vessel, tissue or organ, or, a re-programmed differentiated cell is implanted in the vessel, tissue or organ ex vivo or in vivo. The method also can further comprise implanting the re-programmed differentiated cell in an individual in need thereof.

The invention provides re-programmed differentiated cells made by practicing a method of the invention, wherein the de-differentiated or re-programmed cell is a mammalian cell. The re-programmed differentiated cell can be a mammalian cell, e.g., a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

The invention provides nucleic acids comprising or consisting of (a) a nucleic acid sequence as set forth in SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, or SEQ ID NO:30, or (b) a nucleic acid sequence encoding an amino acid sequence as set forth in SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.

The invention provides polypeptides having an amino acid sequence comprising SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates DRP-GFP fusion protein uptake by primary keratinocytes, as described in detail in Example 1, below.

FIG. 2 illustrates the results of DRP-GFP fusion protein uptake by primary B cells from patients with aggressive (ZAP-POS) or indolent (ZAP-NEG) Chronic Lymphocytic Leukemia (CLL), as described in detail in Example 1, below.

FIG. 3, left panel, illustrates undifferentiated (hESC) H9 (a human ES cell line) (left) and primitive endoderm like cells (PEL cells; center) that spontaneously differentiated; FIG. 3, right panel, illustrates a quantitative proteome comparison of hESC H9 (y-axis) to H9-derived PEL cells (x-axis), as described in detail in Example 1, below.

FIG. 4 illustrates exemplary Reprogramming DRPs (ReD) proteins of this invention, including: an ATF, or Artificial Transcription Factor, domain comprising: a TAD (which in the illustrated exemplary embodiment is a VP-16 transactivation domain—which in one embodiment is replaced by an 11MTAD domain, as discussed in Example 2, below); and AZF, or Artificial Zinc Finger DNA binding domain; and an NLS, or Nuclear Localization Signal (can also be called a Nuclear Localization Peptide, or NLP), which in one embodiment is an SV-20 NLS, as illustrated in this schematic. In the illustrated embodiment the exemplary DRP also comprises a CPP, or cell penetrating peptide, which in the illustrated exemplary embodiment is an R9 CPP. Also illustrated in this Figure is the alternative embodiment of having a DRP of the invention also comprising a TEn, or Transcription/translation enhancing box, which in the illustrated exemplary embodiment is a T7 TEn.

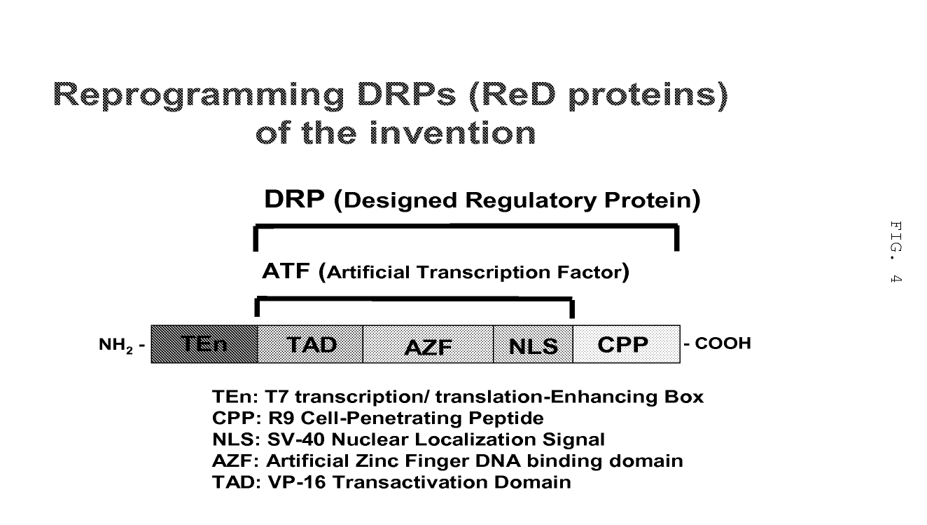

FIG. 5 illustrates how Reprogramming DRPs (ReD) proteins of this invention may target genes to reprogram and/or re-differentiate or de-differentiate cells, although the invention is not limited by any particular mechanism of action. The figure illustrates exemplary Reprogramming DRPs (ReD) proteins of this invention penetrating a cell membrane and a nuclear membrane, entering the nucleus, and activating transcription of a target gene, e.g., the exemplary targets Oct3/4, SOX2, Klf4 and c-Myc are illustrated in this figure.

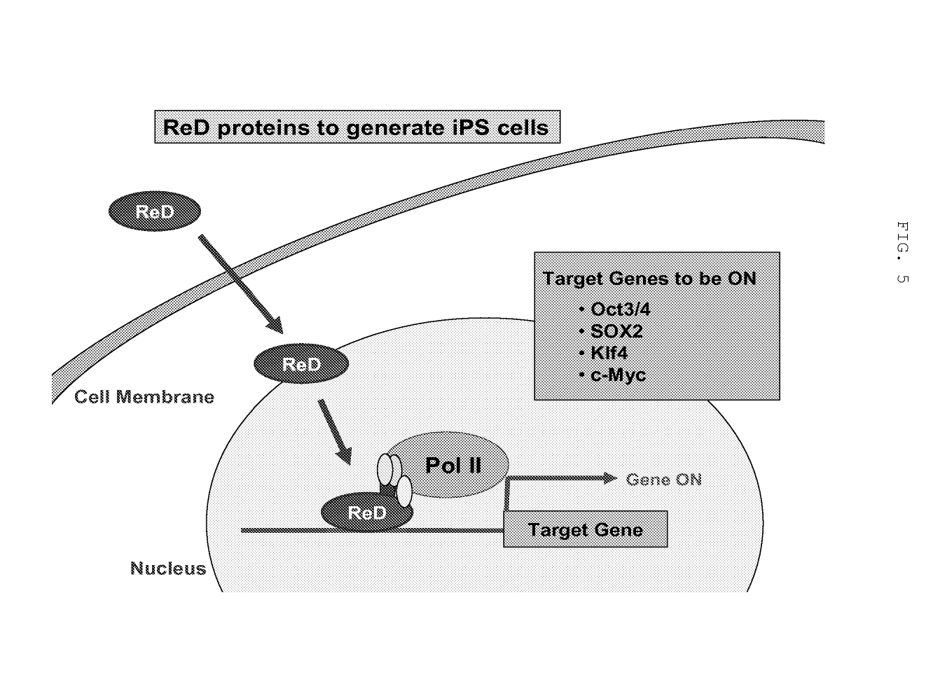

Like reference symbols in the various drawings indicate like elements.

### DETAILED DESCRIPTION

The invention provides compositions and in vitro and ex vivo methods for de-differentiating and/or re-programming mammalian cells. In alternative embodiments, the invention provides compositions comprising mixtures of Designed Regulatory Proteins (DRPs) or Reprogramming DRPs (ReDs) for de-differentiating or re-programming mammalian cells, or mixtures of DRPs or ReDs, or a (one) DRP or ReD that can bind to and activate the transcription of each member of the combination of genes set forth herein, including e.g. the cell programming and differentiation genes, e.g., Oct4, Sox2, Klf4, c-Myc, Lin28, Nanog, and/or Pax5, as described herein The invention also provides compositions and methods for direct reprogramming of a first differentiated phenotype of a cell to a second differentiated phenotype.

The invention provides compositions and in vitro and ex vivo methods for protein-based approaches for manipulating, e.g., de-differentiating or re-programming, mammalian cell phenotypes, e.g., human or animal cell phenotypes, comprising use of Designed Regulatory Proteins (DRPs) or Reprogramming DRPs (ReD) proteins.

DRP or ReD proteins of the invention, or used to practice this invention, can comprise artificial transcription factors, e.g., with 1) one or a plurality of (multiple, e.g., two, three, four, five, six or more) zinc finger binding domains specific for a desired gene, e.g., as described herein; 2) one or a plurality of nuclear localization sequences, e.g., consensus nuclear localization sequences; 3) one or a plurality of cell-penetrating peptide sequences and 4) one or a plurality of transcription activation and/or transcription repression domains. In one embodiment, the DRP or ReD proteins of the invention further comprise a transcription/translation-enhancing box, e.g., a T7 transcription/translation-enhancing box.

In one embodiment, the invention comprises manufacture and design compositions comprising a set of (a plurality of) DRP or ReD proteins of this invention (e.g., a vesicle or nanoparticle) that can specifically target a desired cell type, e.g., a cell targeted for de-differentiation or re-programming. In one embodiment, a protein ligand or antibody is used to specifically target a cell, e.g., to specifically bind to a cell surface molecule or antigen with high affinity and specificity. In one embodiment, the invention provides a DRP or ReD chimeric (e.g., a synthetic or recombinant) protein with the ability to enter cells (e.g., human, mammalian, animal or other cells), go to and penetrate into the nucleus, bind specifically to a targeted gene, and activate or repress transcription of that gene, resulting in the in vitro or ex vivo de-differentiating and/or re-programming of a mammalian cell.

In alternative aspects, DRPs or ReDs of this invention can comprise one or a plurality (e.g., two, three, four or more) domains that enable each of at least four activities: at least one (one or more) protein transduction domain(s); at least one (one or more) nuclear localization signal domain(s); at least one (one or more) DNA-binding domain(s) (e.g., comprising one, two, three, four, five, six or more zinc fingers), and at least one (one or more) transactivation (transcriptional activation) and/or transcriptional repression domain(s).

The compositions and methods of this invention incorporate use of Designed Regulatory Proteins (DRP, also can be ReD), which comprise artificial transcription factors that are fused to protein transduction domains. In alternative embodiments, DRPs used to practice this invention are designed to specifically regulate cell programming and differentiation genes, e.g., Oct4, Sox2, Klf4, c-Myc, Lin28, Nanog, Pax5.

In alternative embodiments, DRPs are added in vitro or ex vivo, e.g., to a medium bathing non-adherent (free) or adherent cells.

In alternative embodiments, DRPs or ReDs used to practice this invention activate or repress expression of key genes, e.g., Oct4, Sox2, Klf4, c-Myc, Lin28, Nanog, Pax5, thus enabling generation (derivation) of a less differentiated cell, including an induced pluripotent stem (iPS) cell, and iPS colonies, without causing any genetic modifications.

In alternative embodiments, DRPs or ReDs used to practice this invention are produced in microbial cells such as bacterial cells, e.g. E. coli, and can be purified at high yields, and can be used at optimized doses.

The iPS cell colonies can be derived at high frequency from different types of human or mammalian somatic cells because they can be taken up by every cell type.

Likewise, any designated differentiated cell type can be generated (derived) using a different set of DRPs or ReDs of this invention. The invention permits cell fate to be controlled in a precise and determinative way without making genetic modifications to the cell.

### Polypeptides and Peptides

In alternative embodiments, the invention provides chimeric polypeptides and peptides for de-differentiating or re-programming a mammalian cell. In alternative embodiments, each DRP or ReD is a chimeric protein comprising: (1) at least one zinc finger DNA binding peptide domain specific for (capable of specifically binding to) a promoter or a transcriptional regulatory region of a gene; (2) at least one nuclear localization peptide (NLP) domain; (3) at least one cell-penetrating peptide (CPP); and, (4) a transcription activation peptide domain and/or a transcription repression peptide domain; and (ii) at least one transcription activation peptide domain of each DRP or ReD chimeric protein can bind to and activate the transcription of at least one of the following genes, and the composition comprises at least one DRP or ReD chimeric protein that can bind to and activate the transcription of each member of a specific combination of genes and/or transcripts, and described herein.

Polypeptides and peptides used to practice the invention can comprise a recombinant protein, a synthetic protein, a peptidomimetic, a non-natural peptide, or a combination thereof. Peptides and proteins used to practice the invention can be recombinantly expressed in vitro or in vivo. The peptides and polypeptides used to practice the invention can be made and isolated using any method known in the art. Polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) including any automated polypeptide synthesis process known in the art.

The DRP or ReD peptides and polypeptides used to practice the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

In alternative embodiments, compositions used to practice the invention, including the DRPs or ReDs described herein, can comprise an oligopeptide, peptide, polypeptide, or protein sequence, or to a fragment, portion, or subunit of any of these and to naturally occurring or synthetic molecules, including, e.g., peptidomimetics and non-natural amino acids. In alternative aspects, DRPs or ReDs used to practice the invention comprise amino acids joined to each other by peptide bonds or modified peptide bonds and may comprise modified amino acids other than the 20 gene-encoded amino acids. The DRP or ReD polypeptides may be modified by either natural processes, such as post-translational processing, or by chemical modification techniques that are well known in the art. Modifications can be designed anywhere in the polypeptide, including the peptide backbone, the amino acid side-chains and the amino or carboxyl termini. The same type of modification can be made in the same or varying degrees at several sites in a given DRP or ReD polypeptide.

In alternative embodiments, a DRP or ReD polypeptide used to practice the invention can have many types of modifications, e.g., modifications including acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, phosphorylation, prenylation, racemization, selenoylation, sulfation and transfer-RNA mediated addition of amino acids to protein such as arginylation. See for example, Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983)). In another embodiment, a DRP or ReD can be glycol-pegylated as described in U.S. Pat. No. 7,405,198; or can be glycosylated as described in U.S. Pat. No. 7,276,475 or U.S. Pat. No. 7,399,613, or U.S. Pat. No. 7,338,933, the later describing O-linked glycosylation of peptides. DRP or ReD proteins used to practice this invention can be acylated as described e.g., in U.S. Pat. No. 7,273,921.

In alternative embodiments, DRP or ReD peptides and polypeptides used to practice the invention can comprise any “mimetic” and/or “peptidomimetic” form. In alternative embodiments, DRP or ReD peptides and polypeptides used to practice the invention can comprise synthetic chemical compounds which have substantially the same structural and/or functional characteristics of natural polypeptides. A mimetic used to practice the invention can be either entirely composed of synthetic, non-natural analogues of amino acids, or, is a chimeric molecule of partly natural peptide amino acids and partly non-natural analogs of amino acids. A mimetic used to practice the invention can also incorporate any amount of natural amino acid conservative substitutions as long as such substitutions also do not substantially alter the mimetic's structure and/or activity.

Routine experimentation will determine whether a synthetic molecule or mimetic is effective for practicing the invention, e.g., has zinc finger DNA binding activity, or nuclear localization peptide activity, or cell-penetrating peptide activity, or transcription activation peptide domain and/or a transcription repression peptide activity. Methodologies detailed herein and others known to persons skilled in the art may be used to select or guide one to choose effective mimetic for practicing the compositions and/or methods of this invention.

Polypeptide mimetic compositions for practicing the invention can comprise any combination of non-natural structural components. In alternative aspects, mimetic compositions for practicing the invention can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids used to practice this invention can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, e.g., under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics that can be used include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

Polypeptides used to practice this invention can comprise signal sequences, i.e., leader sequences, e.g., for secreting a recombinant antibody or inhibitory polypeptide used to practice the invention from a production host cell.

### Antibodies, Therapeutic and Humanized Antibodies

In alternative embodiments, the invention provides that specifically bind to and inhibit or activate a protein transcription factor, e.g., one or a set of transcription factors responsible for maintaining the differentiated phenotype of the differentiated cell, or alternatively for reprogramming the phenotype of a cell. Antibodies can be used in conjunction with the chimeric DRPs of this invention or reprogram and/or to de-differentiate and/or to re-differentiate a cell phenotype.

In alternative embodiments, the invention uses isolated, synthetic or recombinant antibodies that specifically bind to and inhibit or activate a protein transcription factor, e.g., a factor responsible for maintaining a first or a second differentiated phenotype. In alternative embodiments, antibodies used to practice the invention bind to a surface marker, e.g., a polypeptide cell surface marker, present in a de-differentiated or re-programmed cell and not a cell before its de-differentiation or re-programming, e.g., CXCR4, CD10, CD13, CD41a (gpIIbIIIa), CD34, CD56, CD90, CD110, CD117, CD123, CD133, CD135, CD277 and/or CD318.

In alternative aspects, an antibody for practicing the invention can comprise a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. In alternative aspects, an antibody for practicing the invention includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

In alternative embodiments, the invention uses “humanized” antibodies, including forms of non-human (e.g., murine) antibodies that are chimeric antibodies comprising minimal sequence (e.g., the antigen binding fragment) derived from non-human immunoglobulin. In alternative embodiments, humanized antibodies are human immunoglobulins in which residues from a hypervariable region (HVR) of a recipient (e.g., a human antibody sequence) are replaced by residues from a hypervariable region (HVR) of a non-human species (donor antibody) such as mouse, rat, rabbit or nonhuman primate having the desired specificity, affinity, and capacity. In alternative embodiments, framework region (FR) residues of the human immunoglobulin are replaced by corresponding non-human residues to improve antigen binding affinity.

In alternative embodiments, humanized antibodies may comprise residues that are not found in the recipient antibody or the donor antibody. These modifications may be made to improve antibody affinity or functional activity. In alternative embodiments, the humanized antibody can comprise substantially all of at least one, and typically two, variable domains, in which all or substantially all of the hypervariable regions correspond to those of a non-human immunoglobulin and all or substantially all of Ab framework regions are those of a human immunoglobulin sequence.

In alternative embodiments, a humanized antibody used to practice this invention can comprise at least a portion of an immunoglobulin constant region (Fc), typically that of or derived from a human immunoglobulin.

However, in alternative embodiments, completely human antibodies also can be used to practice this invention, including human antibodies comprising amino acid sequence which corresponds to that of an antibody produced by a human. This definition of a human antibody specifically excludes a humanized antibody comprising non-human antigen binding residues.

In alternative embodiments, antibodies used to practice this invention comprise “affinity matured” antibodies, e.g., antibodies comprising with one or more alterations in one or more hypervariable regions which result in an improvement in the affinity of the antibody for antigen; e.g., a targeted transcriptional activating factor, compared to a parent antibody which does not possess those alteration(s). In alternative embodiments, antibodies used to practice this invention are matured antibodies having nanomolar or even picomolar affinities for the target antigen, e.g., a targeted transcriptional activating factor. Affinity matured antibodies can be produced by procedures known in the art.

### Generating and Manipulating Nucleic Acids

In alternative aspects, because the Designed Regulatory Proteins (DRPs) or ReDs used to practice this invention can be used in recombinant form, the invention provides nucleic acids, which themselves can be recombinant, to make them. In other alternative embodiments, the invention provides, e.g., isolated, synthetic and/or recombinant nucleic acids encoding inhibitory nucleic acids (e.g., siRNA, microRNA, antisense, ribozyme) that can inhibit the expression of genes or messages (mRNAs) of one or a set of transcription factors responsible for maintaining a particular phenotype, e.g., a differentiated phenotype. For example, in another alternative embodiment, the invention uses proteins, peptides or nucleic acids that inhibit or suppress the activity of a tumor suppressor gene retinoblastoma-1 (RB1) and/or a p53 tumor suppressor gene (TP53); or, a composition of the invention can comprise a nucleic acid that encodes a large T antigen of the simian virus 40 (SV-40).

In alternative embodiments, nucleic acids of the invention are made, isolated and/or manipulated by, e.g., cloning and expression of cDNA libraries, amplification of message or genomic DNA by PCR, and the like.

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides (e.g., the DRP chimeric proteins or antibodies used to practice this invention) generated from these nucleic acids can be individually isolated or cloned and tested for a desired activity. Any recombinant expression system can be used, including e.g. bacterial, fungal, mammalian, yeast, insect or plant cell expression systems.

Alternatively, nucleic acids used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The invention provides and uses fusion proteins and nucleic acids encoding them. Any polypeptide used to practice this invention (e.g., an antibody or a DRP protein) can be fused to a heterologous peptide or polypeptide, such as a peptide for targeting the polypeptide to a desired cell type, such a first differentiated cell targeted for re-programming to a second differentiated phenotype

In alternative embodiments, a heterologous peptide or polypeptide joined or fused to a protein used to practice this invention can be an N-terminal identification peptide which imparts a desired characteristic, such as fluorescent detection, increased stability and/or simplified purification. Peptides and polypeptides used to practice this invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Nucleic acids or nucleic acid sequences used to practice this invention can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Compounds use to practice this invention include nucleic acids, i.e., oligonucleotides, containing known analogues of natural nucleotides. Compounds use to practice this invention include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Compounds use to practice this invention include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, compounds used to practice this invention include genes or any segment of DNA involved in producing a polypeptide chain (e.g., a DRP protein or an antibody); it can include regions preceding and following the coding region (leader and trailer) as well as, where applicable, intervening sequences (introns) between individual coding segments (exons). “Operably linked” can refer to a functional relationship between two or more nucleic acid (e.g., DNA) segments. In alternative aspects, it can refer to the functional relationship of transcriptional regulatory sequence to a transcribed sequence. For example, a promoter can be operably linked to a coding sequence, such as a nucleic acid used to practice this invention, if it stimulates or modulates the transcription of the coding sequence in an appropriate host cell or other expression system. In alternative aspects, promoter transcriptional regulatory sequences can be operably linked to a transcribed sequence where they can be physically contiguous to the transcribed sequence, i.e., they can be cis-acting. In alternative aspects, transcriptional regulatory sequences, such as enhancers, need not be physically contiguous or located in close proximity to the coding sequences whose transcription they enhance.

In alternative aspects, the invention comprises use of “expression cassettes” comprising a nucleotide sequence used to practice this invention, which can be capable of affecting expression of the nucleic acid, e.g., a structural gene or a transcript (e.g., encoding a DRP or ReD or antibody) in a host compatible with such sequences. Expression cassettes can include at least a promoter operably linked with the polypeptide coding sequence or inhibitory sequence; and, in one aspect, with other sequences, e.g., transcription termination signals. Additional factors necessary or helpful in effecting expression may also be used, e.g., enhancers.

In alternative aspects, expression cassettes used to practice this invention also include plasmids, expression vectors, recombinant viruses, any form of recombinant “naked DNA” vector, and the like. In alternative aspects, a “vector” used to practice this invention can comprise a nucleic acid that can infect, transfect, transiently or permanently transduce a cell. In alternative aspects, a vector used to practice this invention can be a naked nucleic acid, or a nucleic acid complexed with protein or lipid. In alternative aspects, vectors used to practice this invention can comprise viral or bacterial nucleic acids and/or proteins, and/or membranes (e.g., a cell membrane, a viral lipid envelope, etc.). In alternative aspects, vectors used to practice this invention can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice this invention can be stably replicated by the cells during mitosis as an autonomous structure, or can be incorporated within the host's genome.

In alternative aspects, “promoters” used to practice this invention include all sequences capable of driving transcription of a coding sequence in a cell, e.g., a mammalian cell such as a brain cell. Thus, promoters used in the constructs of the invention include cis-acting transcriptional control elements and regulatory sequences that are involved in regulating or modulating the timing and/or rate of transcription of a gene. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

“Constitutive” promoters used to practice this invention can be those that drive expression continuously under most environmental conditions and states of development or cell differentiation. “Inducible” or “regulatable” promoters used to practice this invention can direct expression of the nucleic acid of the invention under the influence of environmental conditions or developmental conditions.

### Antisense Inhibitory Nucleic Acid Molecules

In alternative embodiments, the invention provides antisense or otherwise inhibitory nucleic acid molecules capable of decreasing or inhibiting expression of one or a set of proteins, e.g., one or a set of transcription factors responsible for maintaining the differentiated phenotype of the differentiated cell, or alternatively for reprogramming the phenotype of a cell. Antisense and/or inhibitory nucleic acid molecules can be used in conjunction with the chimeric DRP or ReD proteins of this invention or reprogram and/or to de-differentiate and/or to re-differentiate a cell phenotype.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids.

RNA Interference (RNAi)

In alternative embodiments, the invention uses RNAi inhibitory nucleic acid molecules capable of binding and inhibiting genes and/or messages (transcripts) for one or a set of transcription factors responsible for maintaining the differentiated phenotype of a differentiated cell, or alternatively for reprogramming a cell phenotype, and these RNAi inhibitory nucleic acid molecules can be used in conjunction with the chimeric DRPs or ReDs of this invention.

In one aspect, the invention provides RNAi inhibitory nucleic acid molecules capable of decreasing or inhibiting expression of one or a set of proteins, e.g., one or a set of transcription factors responsible for maintaining the differentiated phenotype of the differentiated cell. In one aspect, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules. For example, in one embodiment, the invention uses inhibitory, e.g., siRNA, miRNA or shRNA, nucleic acids that inhibit or suppress the activity of a tumor suppressor gene retinoblastoma-1 (RB1) and/or a p53 tumor suppressor gene (TP53).

In alternative aspects, the RNAi is about 11, 12, 13, 14, 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, e.g., to silence one or a set of transcription factors responsible for maintaining the differentiated phenotype of the differentiated cell; see, e.g., Shuey (2002) Drug Discov. Today 7:1040-1046. In one aspect, the invention provides methods to selectively degrade an RNA using the RNAi's of the invention. In one aspect, the RNAi molecules of the invention can be used to generate a loss-of-function mutation in a cell. These processes may be practiced in vitro or ex vivo.

In one aspect, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, the invention provides lipid-based formulations for delivering, e.g., introducing nucleic acids of the invention as nucleic acid-lipid particles comprising an RNAi molecule to a cell, see e.g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

Methods for making expression constructs, e.g., vectors or plasmids, from which an inhibitory polynucleotide (e.g., a duplex siRNA of the invention) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, e.g., an NADPH oxidase enzyme coding sequence or transcriptional activation sequence, is inserted between two promoters (e.g., mammalian, viral, human, tissue specific, constitutive or other type of promoter) such that transcription occurs bidirectionally and will result in complementary RNA strands that may subsequently anneal to form an inhibitory siRNA of the invention.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice this invention.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In ones embodiment, the spacer comprises a length of nucleotides of between about 5 to 50 nucleotides. In one aspect, the sense and antisense coding regions of the siRNA can each be on a separate expression vector and under the control of its own promoter.

### Inhibitory Ribozymes

In alternative embodiments, the invention uses ribozymes capable of binding and inhibiting genes and/or messages (transcripts) for one or a set of transcription factors responsible for maintaining the differentiated phenotype of a differentiated cell, or alternatively for reprogramming a cell phenotype, and these ribozymes can be used in conjunction with the chimeric DRP or ReD proteins of this invention.

These ribozymes can inhibit a gene's activity by, e.g., targeting a genomic DNA or an mRNA (a message, a transcript). Strategies for designing ribozymes and selecting a gene-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

### Kits and Instructions

The invention provides kits comprising compositions and methods of the invention, including instructions for use thereof. As such, kits, cells, vectors and the like can also be provided.

For example, in alternative embodiments, the invention provides kits comprising compositions comprising a set of (e.g., the plurality of) Designed Regulatory Proteins (DRPs) or ReDs as set forth herein, (b) a liquid or aqueous formulation of the invention, or (c) a vesicle, liposome, nanoparticle or nanolipid particle of the invention. In one aspect, the kit further comprising instructions for practicing any methods of the invention, e.g., in vitro or ex vivo methods for direct reprogramming of a first differentiated phenotype of a cell to a second differentiated phenotype, or in vitro or ex vivo methods for de-differentiating or re-programming a mammalian cell.

### Formulations

In alternative embodiments, the invention provides compositions for use in in vitro or ex vivo methods (including methods of the invention) for direct reprogramming of a first differentiated phenotype of a cell to a second differentiated phenotype, and for use in in vitro or ex vivo methods (including methods of the invention) for de-differentiating or re-programming a mammalian cells. In alternative embodiments, these compositions comprise a plurality of (a set of) proteins and/or nucleic acids formulated for these purposes, e.g., a plurality of Designed Regulatory Proteins (DRPs) or ReDs formulated in a buffer, in a saline solution, in a powder, an emulsion, in a vesicle, in a liposome, in a nanoparticle, in a nanolipoparticle and the like.

In alternative embodiments, the compositions can be formulated in any way and can be applied in a variety of concentrations and forms depending on the desired in vitro or ex vivo conditions, a desired in vitro or ex vivo method of administration and the like. Details on techniques for in vitro or ex vivo formulations and administrations are well described in the scientific and patent literature.

Formulations and/or carriers of the plurality of Designed Regulatory Proteins (DRPs) or ReDs of this invention that can be used to practice this invention are well known in the art. Formulations and/or carriers used to practice this invention can be in forms such as tablets, pills, powders, capsules, liquids, gels, syrups, slurries, suspensions, etc., suitable for in vitro or ex vivo applications.

The plurality of Designed Regulatory Proteins (DRPs) or ReDs of this invention can be in admixture with an aqueous and/or buffer solution or as an aqueous and/or buffered suspension, e.g., including a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate. Formulations can be adjusted for osmolarity, e.g., by use of an appropriate buffer.

In alternative embodiments, oil-based formulations are used for in vitro or ex vivo application of the compositions (e.g., a set of DRP chimeric proteins) of the invention. Oil-based suspensions can be formulated by suspending the set of chimeric DRP or ReD proteins of the invention in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of hydrophobic compounds; see also U.S. Pat. No. 5,858,401. These formulations can be preserved by the addition of an antioxidant such as ascorbic acid. The formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. Formulations can also contain a buffer, preservative or a coloring agent.

In practicing this invention, the compounds (e.g., formulations) of the invention can comprise a solution of proteins (e.g., a set of DRPs or ReDs of the invention) or nucleic acids dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations used to practice the invention can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent (e.g., Designed Regulatory Proteins) in these formulations can vary widely, and can be selected primarily based on fluid volumes, viscosities and the like, in accordance with the particular mode of in vitro or ex vivo administration selected and the desired results, e.g., for de-differentiating or re-programming a mammalian cell.

The solutions and formulations used to practice the invention can be lyophilized; for example, the invention provides a stable lyophilized formulation comprising a plurality of Designed Regulatory Proteins (DRPs) or ReDs. In one aspect, this formulation is made by lyophilizing a solution comprising a plurality of Designed Regulatory Proteins of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes (see also discussion, below). By using liposomes, particularly where the liposome surface carries ligands specific for target cells, or are otherwise preferentially directed to a specific tissue or organ type, one can focus the delivery of the active agent into a target cells in an in vitro or ex vivo application.

### Nanoparticles, Nanolipoparticles and Liposomes

The invention also provides nanoparticles, nanolipoparticles, vesicles and liposomal membranes comprising compounds used to practice the methods and compositions (e.g., a plurality of DRPs or ReDs) of this invention, e.g., to deliver compositions of the invention to mammalian cells in vitro or ex vivo. In alternative embodiments, these compositions are designed to target specific molecules, including biologic molecules, such as polypeptides, including cell surface polypeptides, e.g., for targeting a desired cell type, e.g., a mammalian cell targeted for de-differentiation or re-programming.

The invention provides multilayered liposomes comprising compounds used to practice this invention, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention (e.g., a plurality of DRPs).

Liposomes can be made using any method, e.g., as described in Park, et al., U.S. Pat. Pub. No. 20070042031, including method of producing a liposome by encapsulating an active agent (e.g., a plurality of DRPs or ReDs), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice this invention comprise a substituted ammonium and/or polyanions, e.g., for targeting delivery of a compound (e.g., a plurality of DRPs or ReDs) used to practice this invention to a desired cell type, as described e.g., in U.S. Pat. Pub. No. 20070110798.

The invention also provides nanoparticles comprising compounds (e.g., a plurality of DRPs) used to practice this invention in the form of active agent-containing nanoparticles (e.g., a secondary nanoparticle), as described, e.g., in U.S. Pat. Pub. No. 20070077286. In one embodiment, the invention provides nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions of the invention (e.g., sets of DRPs or ReDs) to mammalian cells in vitro or ex vivo, as described, e.g., in U.S. Pat. Pub. No. 20050136121.

### Peptide (DRP) Delivery Vehicles

In alternative embodiments, any delivery vehicle can be used to practice the methods or compositions of this invention, e.g., to deliver compositions of the invention (e.g., sets of DRPs or ReDs) to mammalian cells in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition of the invention, wherein a surfactant is associated with a DRP or ReD polypeptide via a noncovalent bond e.g. as described, e.g., in U.S. Pat. Pub. No. 20040151766.

In one embodiment, a covalent conjugate between a poly(alkylene oxide) and a glycosylated or non-glycosylated DRP or ReD is used, where a poly(alkylene oxide) can be conjugated to a DRP or ReD via a glycosyl linking group, and a glycosyl linking group can be interposed between a DRP or ReD and a poly(alkylene oxide). A covalent conjugate can be formed by contacting a DRP or ReD peptide with a glycosyltransferase and a modified sugar donor; the glycosyltransferase transfers the modified sugar moiety to the DRP to form a covalent conjugate; the modified sugar moiety can be a poly(alkylene oxide). See e.g., U.S. Pat. No. 7,416,858.

In one embodiment, a DRP or ReD used to practice this invention can be applied to cells as polymeric hydrogels or water-soluble copolymers, e.g., as described in U.S. Pat. No. 7,413,739; for example, a DRP or ReD can be polymerized through a reaction between a strong nucleophile and a conjugated unsaturated bond or a conjugated unsaturated group, by nucleophilic addition, wherein each precursor component comprises at least two strong nucleophiles or at least two conjugated unsaturated bonds or conjugated unsaturated groups.

In one embodiment, a DRP or ReD used to practice this invention can be applied to cells using vehicles with cell membrane-permeant peptide conjugates, e.g., as described in U.S. Pat. Nos. 7,306,783; 6,589,503. In one aspect, the DRP or ReD itself is conjugated to a cell membrane-permeant peptide. In one embodiment, a DRP or ReD and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver a composition of the invention to a cell, e.g., using any electroporation system as described e.g. in U.S. Pat. Nos. 7,109,034; 6,261,815; 5,874,268.

Products of Manufacture, Implants and artificial organs

The invention also provides products of manufacture comprising cells of the invention, and use of cells made by methods of this invention, including for example implants and artificial organs, bioreactor systems, cell culture systems, plates, dishes, tubes, bottles and flasks comprising cells of this invention. Any implant, artificial organ, bioreactor systems, cell culture system, cell culture plate, dish (e.g., petri dish), cell culture tube and/or cell culture flask (e.g., a roller bottle) can be used to practice this invention.

In alternative embodiments the invention provides a bioreactor, implant, stent, artificial organ or similar device comprising a cell of the invention, or cells made by a method of this invention; for example, including implants as described in U.S. Pat. Nos. 7,388,042; 7,381,418; 7,379,765; 7,361,332; 7,351,423; 6,886,568; 5,270,192; and U.S. Pat. App. Pub. Nos. 20040127987; 20080119909 (describing auricular implants); 20080118549 (describing ocular implants); 20080020015 (describing a bioactive wound dressing); 20070254005 (describing heart valve bio-prostheses, vascular grafts, meniscus implants); 20070059335; 20060128015 (describing liver implants).

### Implanting Cells In Vivo

In alternative embodiments, the methods of the invention also comprise implanting or engrafting the de-differentiated re-programmed cells (of the invention, or made by a method of this invention), or re-programmed differentiated cells (of the invention, or made by a method of this invention) in a vessel, tissue or organ; and in one aspect, comprise implanting or engrafting the re-programmed differentiated cell in a vessel, tissue or organ ex vivo or in vivo, or implanting or engrafting the re-programmed differentiated cell in an individual in need thereof.

Cells can be removed from an individual, treated using the compositions and/or methods of this invention, and reinserted (e.g., injected or engrafted) into a tissue, organ or into the individual, using any known technique or protocol. For example, de-differentiated re-programmed cells, or re-programmed differentiated cells, can be re-implanted (e.g., injected or engrafted) using microspheres e.g., as described in U.S. Pat. No. 7,442,389; e.g., in one aspect, the cell carrier comprises a bulking agent comprising a plurality of round and smooth polymethylmethacrylate microparticles preloaded within a mixing and delivery system and an autologous carrier comprising these cells. In another embodiment, the cells are readministered to a tissue, an organ and/or an individual in need thereof in a biocompatible crosslinked matrix, as described e.g., in U.S. Pat. App. Pub. No. 20050027070.

In another embodiment, the cells of the invention (e.g., cells made by practicing the methods of this invention) are readministered (e.g., injected or engrafted) to a tissue, an organ and/or an individual in need thereof within, or protected by, a biocompatible, nonimmunogenic coating, e.g., as on the surface of a synthetic implant, e.g., as described in U.S. Pat. No. 6,969,400, describing e.g., a protocol where a DRP or ReD can be conjugated to a polyethylene glycol that has been modified to contain multiple nucleophilic groups, such as primary amino or thiol group.

In one embodiment, the cells of the invention (e.g., cells made by practicing the methods of this invention) are readministered (e.g., injected or engrafted) to a tissue, an organ and/or an individual in need thereof using grafting methods as described e.g. by U.S. Pat. Nos. 7,442,390; 5,733,542.

The invention will be further described with reference to the following examples; however, it is to be understood that the invention is not limited to such examples.

### EXAMPLES

### Example 1

The protocols presented herein can be used to demonstrate that the compositions (e.g., sets of DRPs or ReDs of the invention) and methods of the invention are effective for re-programming or de-differentiating mammalian cells.

The invention provides sets of DRPs or ReDs of the invention, including for example 6-finger Designed Regulatory Protein (DRP) or ReD activators of the human Oct4, Sox2, Klf4, c-Myc, Lin28, and Nanog genes. Dose-response curves will be constructed testing the Oct4 and Sox2 DRPs in a matrix, using ES cell-like colony formation and qRT-PCR for Dnmt3b and Utf1 mRNAs as read-outs. The most effective combined dose of Oct4xSox2 will be chosen and used in a second matrix against each of the other 4 DRPs. The most effective combined dose of 3 DRPs will be chosen and used in a third matrix against the other 3 DRPs. This design will be iterated until the “Best Mix” of all 6 factors has been found.

A 9-finger DRP will be constructed with 3-finger specificity for Oct4, Sox2, and the 3^{rd} most potent gene to determine if a monomolecular agent is effective.

Cells can be grown under feeder-free, serum-free conditions. The best mix can be used to derive iPS cell lines (3 each from 12 healthy volunteers) for comparison to human ES cell lines H1 and H9 (see e.g., Thomson (1998) Science 282 (5391):1145-1147), using genome-wide proteomics, ability to form embryoid bodies that stain for antigen markers of all 3 germ layers, and for teratoma formation.

Repression of somatic cell identity will be tested to evaluate whether this repression can increase reprogramming efficiency by treating primitive endoderm-like cells (PELs), derived from ES cells using our standard protocol, with the best mix augmented by a DRP that represses GATA6 (GATA binding protein 6).

To test whether somatic cell identity mutations are required to form iPS cells, iPS cells will be derived from PELs; and then it will be determined if they can differentiate back to PELs using a standard ES cell differentiation protocol. Failure to re-form PELs would indicate a mutation in a PEL-cell identity gene had occurred.

Clinical grade iPS cell lines will be derived from patients with Chronic Lymphocytic Leukemia (CLL). Twelve (12) iPS cell lines each from primary fibroblasts of patients with Chronic Lymphocytic Leukemia (CLL; aggressive and indolent) and healthy volunteers will be derived using the best mix. Afterwards, this approach will be extended to keratinocytes and B cells. The best mix will be tried, but if it is not effective, then the iterative process will be repeated as described above to develop a best “mix-b” for B cells and a best “mix-k” for keratinocytes. The best mix will be augmented with DRP repressors of keratinocyte or B cell transcription factors to destabilize cell identity (e.g., E2A in B cells).

Every iPS cell line will be tested for its ability to make all 3 germ layers in embryoid bodies. Three iPS cell lines derived from fibroblasts and three from B cells of a patient cohort with aggressive CLL will be differentiated into hematopoietic stem cells and transplanted into immune-deficient mice. The mice will be observed for development of CLL symptoms.

The mouse CLL disease model will be assessed at to whether it accurately reflects the corresponding human disease. If no disease occurs, then CLL may not have a genetic basis or it may be a non-cell autonomous trait. If the disease potential of B cell-derived iPS cell lines is high and fibroblast-derived iPS cell lines is low, and both come from the same CLL patient, then CLL may be caused by somatic mutations in the B cell lineage.

Design and Construction of DRP-encoding DNA. Artificial zinc finger (AZP) DNA binding domains (DBD) that can bind to selected 19-bp sequence targets in the promoter of genes are rationally designed using a recognition code table.

For example, the following are exemplary Reprogramming DRP (ReD) proteins that can be used to practice the invention; in alternative embodiments, the equivalent human promoter is used in place of the mouse promoter:

Reprogramming DRP (ReD protein)

| Protein | Target Gene |
|---|---|
| ReD-1 | mouse Oct4 promoter |
| ReD-2 | mouse Oct4 promoter |
| ReD-3 | mouse SOX2 promoter |
| ReD-4 | mouse SOX2 promoter |
| ReD-5 | mouse Klf-4 promoter |
| ReD-6 | mouse Klf-4 promoter |
| ReD-7 | mouse c-Myc promoter |
| ReD-8 | mouse c-Myc promoter |
| ReD-9 | mouse Nanog promoter |
| ReD-10 | mouse Nanog promoter |

The DNA encoding the AZP is constructed by gene assembly. The DNA encoding the AZP was cloned into pTriEX-3 (Novagen, EMD Chemicals Inc., an Affiliate of Merck KGaA, Darmstadt, Germany) containing a protein transduction domain (PTD) 9-mer of arginine (R9); a nuclear localization signal (NLS) (can also be called a nuclear localization peptide, or NLP) from the SV40 large T antigen; an trans-effect domain (TED); and a FLAG epitope tag.

In one embodiment, to boost protein yields in E. coli about 50-fold and enhance protein transduction, a TEnBox (T7 enhancer box) can be placed at the amino terminus. In one embodiment, as an activation domain, herpes simplex virus (HSV) VP-16 activation domain (residues 415-490) is used.

In one embodiment, for repression domains, a Krüppel-associated box (KRAB) domain of KOX1 (residues 1-75) is introduced into the DRP construct. In one embodiment, the Krüppel-associated box (KRAB) domain of KOX1, residues 1 to 75, e.g. as described by Margolin (1994) Proc. Natl. Acad. Sci. USA 91:4509-4513, and Tachikawa (2004) Proc. Natl. Acad. Sci. USA 101:1525-15230, is used as a repression domain.

In alternative embodiments, eleven copies of the last five amino acids derived from the C-terminal transcription activation domain of β-catenin (FDTDL) or an SRDX domain from Arabidopsis thaliana SUPERMAN protein are introduced into the DRP construct for transactivation or repression domains, respectively. In one embodiment, DRPs with domains derived from human β-catenin are used. In one embodiment, a “minimal transactivation domain”, or MTAD, residues 695-781 from human β-catenin (see e.g., Hecht (1999) J. Biol. Chem. 274:18017-18025) can be used. This can result in, e.g., a 3-fold induction of a VEGF-A protein (see e.g., Tachikawa (2004) supra). One copy of the MTAD motif, FDTDL, of β-catenin may not activate transcription, however, three to six or more tandem repeats of the motif can activate transcription, e.g., can induce 2- and 4-fold increases in some embodiments and uses; for example, see e.g. Tachikawa (2004) supra, describing how eleven (11) copies of the MTAD motif caused the DRP to induce VEGF-A protein 15-fold; the activation potency was nearly twice that of a VP-16 transactivation domain.

This invention provides technology that enables cell fate to be precisely controlled and characterized. By practicing the compositions and methods of this invention, cell fate can be controlled using sets of Designed Regulatory Proteins (DRPs) of the invention; these sets of DRPs can specifically activate or repress target genes without modifying DNA, e.g., without modifying a cell's chromosomal nucleic acid.

Cells will be characterized using genome-wide proteomics to provide the identification and quantitative measures of several thousand proteins in cell extracts and secretions. From these characterizations we will develop antibody biomarkers that can be used to score and enrich specific cell types.

Embryonic stem (ES) cells will be differentiated into primitive endoderm-like (PEL) cells using established methods and then sets of DRPs of the invention will be used to derive induced pluripotent stem (iPS) cell lines from them. These exemplary sets of DRPs of the invention will include activators of Oct4, Sox2, and Klf4 (or Nanog) with or without c-Myc plus a DRP repressor of GATA6 (GATA binding protein 6).

A protocol to derive iPS cell lines can be developed using primary keratinocytes, e.g., from a mouse. To assess the effectiveness of a DRP of the invention, the iPS cells can be compared to ES cells using proteomics and RT-PCR, and their ability to form PEL cells can be observed. Sets of DRPs of the invention can be tested for their ability to cause differentiation into PEL cells by treating ES cells with sets of DRPs that have reciprocal activities to those mentioned above. Proteomics can be used to measure changes during the transitions from ES cells to PEL cells to iPS cells, testing for “hysteresis” in splicing, post-translational modifications (e.g., phosphorylation), protein abundance, and the secretome. This screening can be done with either mouse or human cells, or first mouse and then human cells.

Exemplary sets of DRPs of the invention also will be used to derive clinical-grade iPS cells lines from B cells of patients with chronic lymphocytic leukemia (CLL) to validate this aspect of the invention, and to provide a predictive model for the most common form of human adult leukemia. Exemplary sets of DRPs of the invention also will be used to derive iPS cell lines from exocrine and endocrine pancreas cells.

In alternative embodiments, the invention provides sets of Designed Regulatory Proteins (DRPs) in the form of artificial transcription factors that are designed to specifically activate re-programming genes such as Oct4, Sox2, Klf4, c-Myc, Lin28, Nanog genes.

Because DRPs of this invention are fused to protein transduction domains (e.g., at least one cell-penetrating peptide (CPP), and at least one nuclear localization peptide (NLP) domain), they are taken up (internalized) by mammalian cells.

In alternative embodiments, DRPs of the invention are produced (as recombinant proteins) in bacterial, fungal, mammalian, yeast, insect or plant cells; e.g., in one aspect they are produced in E. coli. Recombinantly made DRPs of the invention can be purified at high yields and can be used at optimized doses.

We previously showed that this approach successfully caused the correct isoform ratios of VEGF-A to be secreted by cultured human cells (see reference 6, below). DRPs should produce iPS colonies at high frequency unless reprogramming requires mutations or stochastic down-regulation of somatic cell identity genes. If the frequency is not improved DRPs can be added that repress key transcription factors to destabilize the cell identity of the somatic cell.

In one embodiment, valproic acid (VPA), a histone deacetylase inhibitor, is added to improve reprogramming efficiency; which can be by more than 100-fold without introduction of the oncogene c-Myc (see reference 7, below). VPA can be added to obtain iPS cells with higher efficiency.

In one embodiment, mouse primitive endoderm-like (PEL) cells, B cells, keratinocytes, and fibroblasts are re-programmed. In one embodiment, iPS cell lines are derived from exocrine pancreatic cells that secrete pancreatic juice containing digestive enzymes such as trypsin, chymotrypsin and pancreatic lipase, and endocrine pancreatic cells that produce several important hormones including insulin, glucagon and somatostatin. These protocols and screening methods can be repeated with mouse and/or human cells.

In one embodiment, methods of the invention comprise identifying and/or isolating a de-differentiated or re-programmed cell by using an antibody that specifically binds to a polypeptide cell surface marker, e.g., a biomarker, present in the de-differentiated or re-programmed cell and not the cell before de-differentiating or re-programming. The method of the invention can use any biomarker that distinguishes a stem cell from a derivative, e.g., a gene transcript (mRNA) can be a biomarker; and in one aspect, the invention uses nucleic acid microarrays to characterize cells by identifying what set of markers, e.g., genes or expressed transcripts, before de-differentiating or re-programming and/or after de-differentiating or re-programming.

Changes in gene expression also can be detected using reporters, or in another aspect cell surface biomarkers are detected by antibodies; use of antibodies sometimes is preferred as a means to select or exclude specific cell types within mixed populations. Any antibody-based biomarkers that can label and/or separate stem cells from differentiated cell types can be used to practice this invention.

Antigen/antibody biomarkers can be developed directly using mass spectrometry to identify plasma membrane proteins in differentiated or re-programmed cells. Desired cell types, e.g., the differentiated or re-programmed cells, can be prepared in large, pure batches, e.g., including embryonic stem (ES) cells or primitive endoderm-like (PEL) cells derived from ES cells. Other cell types also can be used.

Use of biomarkers will enable the use of autologous stem cell transplants. Because transplantation of differentiated cells or tissues derived from stem cells carries the risk that proliferating stem cells remain that could produce teratomas or cause other complications, stem cell antibody biomarkers could provide quality controls to quantify the contamination of tissue transplants by stem cells, e.g., hES or hMS cells. Stem cell antibody biomarkers can also be used to removal stem cells by negative sorting or by selective toxicity. Biomarkers that specifically recognize differentiated progenitors such as PEL cells can be used as quality controls to characterize the purity of the culture or to permit positive sorting so that purity can be increased.

In one embodiment, the invention provides clinical grade iPS cell lines. To generate (derive) clinical grade iPS cell lines, six (6)-finger DRP activators of the mouse and human Oct4, Sox2, Klf4, c-Myc, Lin28, and Nanog genes are constructed. Dose-response curves with primary keratinocytes can be constructed testing the Oct4 and Sox2 DRPs in a matrix, using ES cell-like colony formation and qRT-PCR for Dnmt3b (DNA (cytosine-5-)-methyltransferase 3 beta) and Utf1 (undifferentiated embryonic cell transcription factor 1) mRNAs as read-outs. The most effective combined dose of Oct4 and Sox2 can be chosen and used in a second matrix against each of the other 4 DRPs (Klf4, c-Myc, Lin28, and Nanog). The most effective combined dose of 3 DRPs is chosen and used in a third matrix against the other 3 DRPs. This design is iterated until the “Best Mix” of all 6 DRP factors has been found. In one embodiment, a 9-finger DRP with 3-finger specificity for Oct4 and Sox2 is constructed, and the 3rd most potent gene to see if a monomolecular agent is effective.

Mouse keratinocyte cells can be grown under feeder-free, serum-free conditions. The Best Mix can be used to derive iPS cell lines for comparison to mouse ES cell lines using genome-wide proteomics, ability to form embryoid bodies that stain for antigen markers of all 3 germ layers, and for teratoma formation. It can be tested to determine whether repression of somatic cell identity can increase reprogramming efficiency by treating primitive endoderm-like cells (PELs), derived from ES cells using this standard protocol, with the Best Mix augmented by a DRP that represses GATA6.

One embodiment comprises testing whether somatic cell identity mutations are required to form iPS cells by e.g. deriving (generating) iPS cells from PELs, and then trying to differentiate (reprogram) them back to PELs, e.g.,. using a standard ES cell (reprogramming) differentiation protocol. Failure to re-form PELs would suggest that mutations in PEL-cell identity genes had occurred. Once it is confirmed that the iPS cells derived (generated) from keratinocytes are able to differentiate into PEL cells, the cells will be further (reprogrammed) differentiated back into keratinocytes. For other somatic cell types, sets of DRPs of the invention that can repress cell-identity genes comprise Pax-5 for B-cells, p63 for keratinocytes and Ptf1a or Pax4 for pancreas.

Development of biomarkers. In one embodiment, the human proteome, encompassing more than 20,000 proteins as identified directly by mass spectrometry, is used to develop biomarkers useful for practicing this invention, e.g., for identifying and/or isolating differentiated cells, re-differentiated (reprogrammed) cells and/or undifferentiated cell. In one embodiment, the invention uses biomarkers for cells generated using compositions and methods of this invention, e.g., pluripotent cells, including hES cells, mES cells, and murine embryonal carcinoma cells, and cells generated using compositions and methods of this invention, including re-differentiated (reprogrammed) cells such as hPEL cells, mPEL cells, and embryoid bodies (EBs).

For example, the proteome of fractionated macrophages can be used to determine the subcellular distribution of proteins and quantifying how they change in response to one or more DRPs. Similar protocols can be used to isolate plasma membrane proteins or identify a re-differentiated (reprogrammed) cell, a differentiated cell and/or an undifferentiated cell. Protein biomarkers can be used as antigens to obtain monoclonal antibodies.

In one embodiment, for validation antibodies directed a novel surface biomarker first can be used to quantify the purity of a cell culture, e.g., a pluripotent cell culture, including hES or mES cell cultures, or ES and PE cell cultures, e.g., during a plating cycle using, e.g., fluorescence-activated cell sorting (FACS) and/or immunofluorescence or similar methods.

In one embodiment, antibodies able to identify surface biomarkers are used for positive and/or negative selections by FACS. Cell morphology, reporter cell lines, and RT-PCR can be used to quantify the expression of cell identity genes, e.g., including nanog and GATA-6.

Development of a CLL disease model. In one embodiment, iPS cell lines are derived from keratinocytes and B cells of patients with aggressive or indolent Chronic Lymphocytic Leukemia (CLL) and healthy volunteers. The Best Mix (see above) can be used; and if it (the Best Mix) is not effective then the iterative process is repeated, as discussed above, to develop a “Best Mix-b” for B cells. The Best Mix can be augmented with DRP repressors of B cell transcription factors to destabilize cell identity (e.g., PAX5). iPS cell lines can be tested for their ability to form all 3 germ layers in embryoid bodies. iPS cell lines derived from B cells of a patient cohort with aggressive CLL can be differentiated into hematopoietic stem cells and transplanted into immune-deficient mice, which then are observed for development of CLL symptoms. The mouse CLL disease model can be used as a model for the corresponding human disease. If no disease occurs, then CLL may not have a genetic basis or it may be a non-cell autonomous trait. If the disease potential of B cell-derived iPS cell lines is high and keratinocyte-derived iPS cell lines is low, and both come from the same CLL patient, then CLL may be caused by somatic mutations in the B cell lineage.

### Designs and Methods

Design and Construction of DRP-encoding DNA. Artificial zinc finger protein (AZP) DNA binding domains (DBD) that can bind to selected 19-bp sequence targets in the promoter of genes are rationally designed using a recognition code table (see reference 6, below). The DNA encoding the AZP is constructed by gene assembly. The DNA encoding the AZP is cloned into pTriEX-3 (Novagen, EMD Chemicals Inc., an Affiliate of Merck KGaA, Darmstadt, Germany) containing a protein transduction domain (PTD) 9-mer of arginine (R9); a nuclear localization signal (NLS) (or nuclear localization peptide, or NLP) from the SV40 large T antigen; a trans-effect domain (TED); and a FLAG epitope tag.

To boost protein yields in E. coli about 50-fold and enhance protein transduction we place a TEnBox (T7 enhancer box) at the amino terminus. As an activation domain, herpes simplex virus (HSV) VP-16 activation domain (residues 415-490) is used. For repressor domains, a Krüppel-associated box (KRAB) domain of KOX1 (residues 1-75) is introduced into the DRP construct. Eleven copies of the last five amino acids derived from the C-terminal transcription activation domain of β-catenin (FDTDL) or an SRDX domain from Arabidopsis thaliana SUPERMAN protein can be introduced into the DRP construct for transactivation or repression domains, respectively. The pTriEX-3 constructs are expressed in E. coli Rosetta (DE3) pLac I (Novagen). The protein is chromatographically purified to >95% homogeneity as judged by SDS/PAGE. Electrophoretic Mobility Shift Assay (EMSA) binding reactions are carried out at 4° C. for minutes. The gel is blotted and then visualized using LIGHTSHIFT™ Chemiluminescent Kit (Pierce, Thermo Fisher Scientific, Rockford, Ill.) according to the manufacture's instructions. A total of 1×104 stem cells per well are plated onto a 96-well tissue culture plate and incubated at 37° C. for 24 h. Subsequently, DRP solution in OPTI-MEM I™ Reduced Serum Medium (Gibco, Invitrogen, Carlsbad, Calif.) is added to each well and incubated at 37° C. for 5 h. PCR amplification reaction is conducted for 35 cycles at 94° C. for 30 s and at 60° C. for 30 min. PCR amplification of the housekeeping gene, glyceraldehydes-3-phosphate dehydrogenase (GAPDH), is performed to allow normalization between samples.

Isolation and growth of PEL cells. The human ES cell line H9 is maintained in feeder-free culture in mouse embryonic fibroblast-conditioned media. Human ES cells are plated on MATRIGEL™ (BD Biosciences, San Jose, Calif.)-coated plates; the media is changed daily. When the cells reach 90% confluence, human ES cell-derived primitive endoderm-like PEL cells are dissociated with 200 U collagenase IV per ml (Invitrogen, Carlsbad, Calif.) for 5 minutes (mins). Collagenase IV was removed and wells were rinsed with DMEM to collect the PEL cells. Supernatant containing the PEL cells was centrifuged at 900 g for 5 min and the cell pellet was re-suspended in 10% FBS media and transferred to fresh MATRIGEL™-coated wells. The media was changed every other day and PEL cells were passaged when confluent in the same manner as for human ES cells.

In vitro Differentiation of mouse ES cells to keratinocytes. Mouse embryonic stem cell line 129 is cultured and induced to differentiate as described in reference 8, below. The K14 positive basal keratinocyte cells derived from ES cells are separated by anti-integrin alpha6 antibody.

In vitro Differentiation of mouse ES cells to B-cells. Mouse embryonic stem cell line 129 is cultured and induced to differentiate as described in reference 9, below. The B-cells derived from ES cells are separated by magnetic bead-based MACS™ B-cell Isolation Kit (Miltenyi Biotec GmbH, Germany).

In vitro Differentiation of mouse ES cells to β-cells. Mouse embryonic stem cell line 129 is cultured and induced to differentiate as described in reference 10, below. The β-cells derived from ES cells are isolated based on ability to secrete insulin.

Preparation of clinical-grade iPS cells. Cells will be derived and grown in PLURIEXP™ feeder-free/serum-free (FF/SF) medium (Horizon Stemtech Corporation).

Protein extraction and digestion for mass spectrometry: A standard cell lysis buffer contains 2% RAPIGEST™ (Waters Corp., Milford, Mass.) in 10 mM HEPES buffer. Benzonase is added to degrade DNA and RNA to obtain a clear solution. The extracted proteins are treated with 2 mM TCEP (Tris(2-carboxyethyl)phosphine) at 37° C. for 30 minutes to reduce all of the disulfide bonds. Then 5 mM iodoacetamide (IAA) is added to and the sample is incubated in the dark at 37° C. for 30 minutes to alkylate all of the sulfhydryl groups. The proteins are digested by adding trypsin (1:50) and shaking at 37° C. overnight. The completion of digestion is checked by silver stained gel. The digested peptides are acidified by adding TFA to a final concentration of 1% (v/v, pH=1.5) to break down RAPIGEST™. Samples are incubated at 4° C. overnight and then centrifuged at 16,100 g for 15 minutes. Supernatant is taken for 2D-LC-MS/MS analysis.

iTRAQ™ (ITRAQ™) (Invitrogen) labeling: Digested peptides are labeled with iTRAQ™ by adding 1:1 (v/v) iTRAQ™ reagents dissolved in 100% isopropanol (IPA). A set of 4 samples can be labeled using the 4 iTRAQ™ reagents, 114, 115, 116, and 117. Samples are incubated at room temperature for 1 hour. Reactions are stopped by adding Tris buffer to a final concentration of 20 mM. Labeled samples are pooled together and RAPIGEST™ is precipitated by acidifying the solution. Supernatant is taken for 2D-LC-MS/MS analysis.

Phosphopeptide enrichment. Phosphopeptides are enriched using TiO₂ affinity chromatography. 5 uM TiO₂ beads are packed into an empty spin column and washed by water followed by 1% TFA. Digested peptides or iTRAQ™ labeled peptide mixtures are added to the column and incubated at room temperature for 15 minutes. Unbound peptides are spun off by centrifuging. Non-specific binding peptides are washed off by 1% TFA in 70% IPA solution (pH=1.5). Phosphopeptides are eluted from TiO₂ by 50 mM ammonium phosphate in 50% IPA. Eluted peptides are acidified by 3% (v/v) formic acid to a final pH of 3 and analyzed by 2D-LC-MS/MS.

Chromatography. Automated 2D nanoflow LC-MS/MS is used. An AGILENT 1100™ HPLC system (Agilent Technologies, Wilmington, Del.) was used to deliver a flow rate of 300 mL min⁻¹ to the mass spectrometer through a splitter. Chromatographic separation was accomplished using a 3 phase capillary column. Peptides were first eluted from the RP1 column to the SCX column using a 0 to 80% acetonitrile gradient for 150 minutes. The peptides were fractionated by the SCX column using a series of salt gradients (10 mM, 15 mM, 20 mM, 25 mM, 30 mM, 35 mM, 40 mM, 45 mM, 50 mM, 60 mM, 70 mM, 80 mM, 90 mM, 100 mM, 150 mM, 200 mM, 1 M ammonium acetate for 20 minutes), followed by high resolution reverse phase separation using an acetonitrile gradient of 0 to 80% for 120 minutes. It was found that a 3D run can provide significantly more resolving power but at the cost of a longer separation time. For 3D, fractions are eluted with acetonitrile from RP1 in 10% increments then perform the salt elutions as described above but with a resolving gradient for RP2 of acetonitrile equal to the gradient used to elute from RP1.

Electrospray. A custom nano-electrospray device was manufactured and used for the experiments described. 1500 volts was used for the electrospray. Other voltages and other flow rates beyond a standard 250 nL/min can be tested to determine whether they can increase the number of peptides identified.

Mass spectrometry. All of our analyses were performed using LTQ™ linear ion trap tandem mass spectrometers (Thermo Electron Corporation, San Jose, Calif.) employing automated, data-dependent acquisition. As a final purification step, gas phase separation in the ion trap was employed to separate the peptides into 3 mass classes prior to scanning; the full MS scan range of 300-2000 m/z was divided into 3 smaller scan ranges (300-800, 800-1100, and 1100-2000 Da) to improve the dynamic range. Each MS scan was followed by 4 MS/MS scans of the most intense ions from the parent MS scan. PQD (Pulsed-Q Dissociation) was used to enable the detection of MS/MS reporter ions (m/z=114, 115, 116, and 117) from iTRAQ™ which are normally not detectable on ion trap mass spectrometers. An extra PQD scan is added after each CID (Collision Induced Dissociation) MS/MS scan of the same precursor ion. The iTRAQ™ reporter ion (114-117) intensities are used for relative quantitation while both CID and PQD fragmentation peaks are used for peptide identification. Raw data were extracted and searched using SPECTRUMMILL™ (Agilent Technologies, Wilmington, Del., version A.03.02™). The empirical False Discovery Rate (FDR) was calculated by searching the data against a concatenated forward-reverse database. The FDR of our filtering criteria is 0.1% spectra, and 1% protein. Proteins with shared peptides are grouped together into protein groups. iTRAQ™ intensities were calculated by summing the peptide iTRAQ™ intensities from each protein group. Peptides shared among different protein groups were removed before quantitation.

### Results

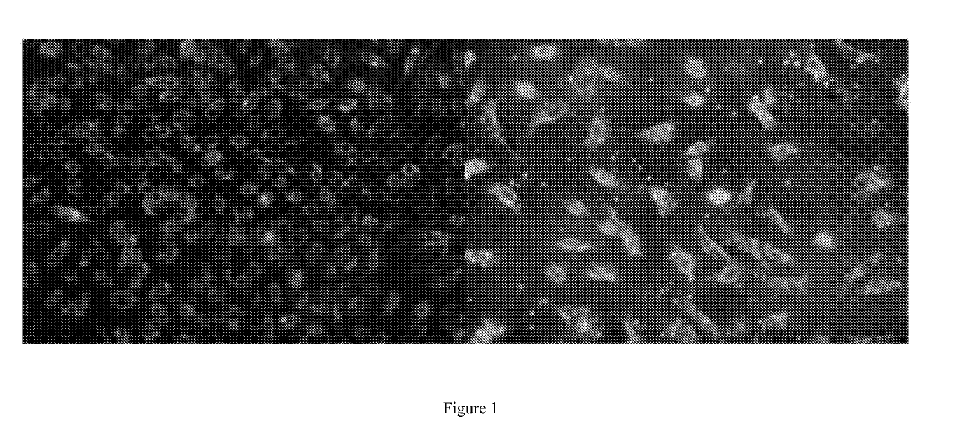

All of the primary cell types from which iPS cell lines will be derived readily take up DRP-GFP (Green Fluorescent Protein) fusion protein indicating that they will also take up the reprogramming DRPs, as illustrated in FIG. 1. FIG. 1 illustrates DRP-GFP fusion protein uptake by primary keratinocytes. The left panel illustrates cells stained primarily in the cytoplasm because the protein transduction domain (PTD) used wasn't effective for this cell type; in contrast, the right panel illustrates that good nuclear staining is observed when a different PTD was used.

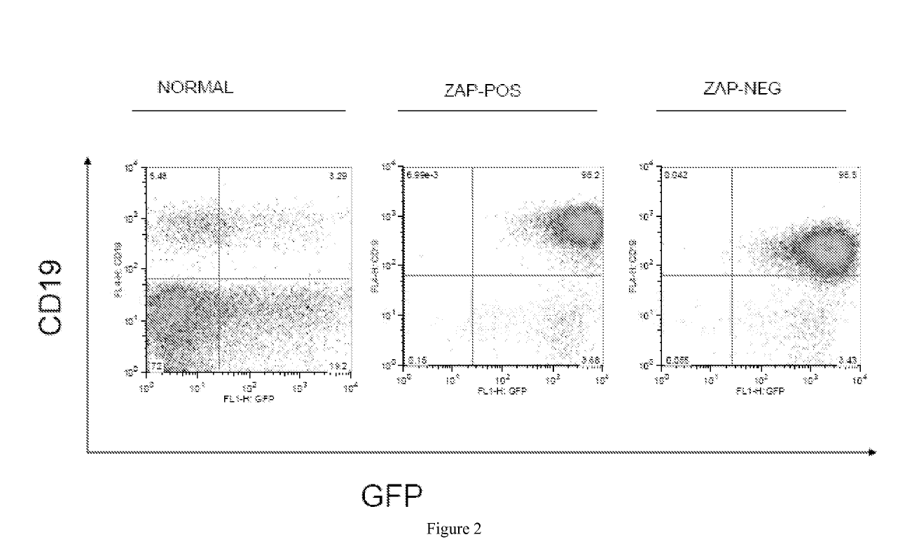

FIG. 2 illustrates the results of DRP-GFP fusion protein uptake by primary B cells from patients with aggressive (ZAP-POS) or indolent (ZAP-NEG) Chronic Lymphocytic Leukemia (CLL). CD19 is a B cell lineage marker; CLL cells express more CD19 than normal cells, with aggressive CLL expressing relatively more CD19 than indolent CLL. Higher GFP levels indicates a greater amount DRP-GFP fusion protein uptake.

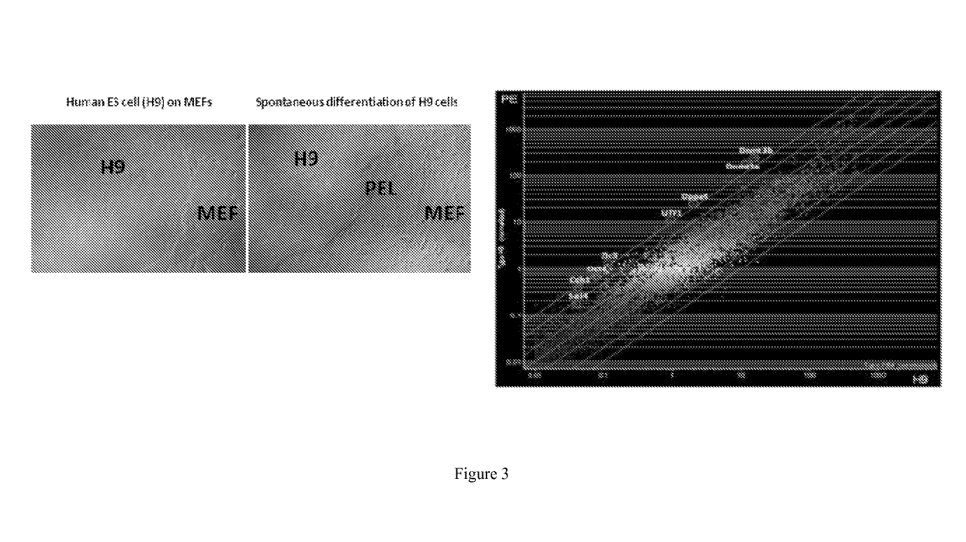

FIG. 3, left panel, illustrates undifferentiated (hESC) H9 (a human ES cell line) (left) and primitive endoderm like cells (PEL cells; center) that spontaneously differentiated. Human embryonic stem cells (hES cells) spontaneously form primitive endoderm-like cells (PEL cells) at the margins of the colony. The PEL cells can be purified using collagenase and cultured for several passages. Differentiation of an entire ES cell colony into PEL cells can be rapidly induced by treatment with 4β-12-O-tetradecanoylphorbol-13-acetate (TPA); GATA6 gene induction is one of the earliest changes.

Several thousand proteins can be studied in extracts from hES cells. 4,181 proteins were identified and quantified at a protein-level FDR of 1.6%; 230 proteins had significantly higher protein levels in undifferentiated cells. The data can be mined further for changes in splice isoforms and post-translational modifications [11,12] to provide a rich profile of cell identity. Prognostic peptides can be used to elicit monoclonal antibodies for use as biomarkers.

FIG. 3, right panel, illustrates a quantitative proteome comparison of hESC H9 (y-axis) to H9-derived PEL cells (x-axis).

### REFERENCES

1. Takahashi, K. and S. Yamanaka, Induction of Pluripotent Stem Cells from Mouse Embryonic and Adult Fibroblast Cultures by Defined Factors. Cell, 2006. 126(4): p. 663-676.
2. Takahashi, K., et al., Induction of Pluripotent Stem Cells from Adult Human Fibroblasts by Defined Factors. Cell, 2007. 131(5): p. 861-872.
3. Okita, K., T. Ichisaka, and S. Yamanaka, Generation of germline-competent induced pluripotent stem cells. Nature, 2007. 448(7151): p. 313-317.
4. Yu, J., et al., Induced Pluripotent Stem Cell Lines Derived from Human Somatic Cells. Science, 2007. 318(5858): p. 1917-1920.
5. Xie, D., et al., An engineered vascular endothelial growth factor-activating transcription factor induces therapeutic angiogenesis in ApoE knockout mice with hindlimb ischemia. Journal of Vascular Surgery, 2006. 44(1): p. 166-175.
6. Tachikawa, K., et al., Regulation of the endogenous VEGF-A gene by exogenous designed regulatory proteins. Proceedings of the National Academy of Sciences, 2004. 101(42): p. 15225-15230.
7. Huangfu, D., et al., Induction of pluripotent stem cells by defined factors is greatly improved by small-molecule compounds. Nat Biotechnol, 2008. June 22. [Epub ahead of print]
8. Coraux, C., et al., Reconstituted skin from murine embryonic stem cells. Curr Biol, 2003. 13(10): p. 849-53.
9. Gutierrez-ramos, J. C., et al., In vitro differentiation of embryonic stem cells into lymphocyte precursors able to generate T and B lymphocytes in vivo. Proceedings of the National Academy of Sciences, 1992. 89(19): p. 9171-5.
10. Lumelsky, N. et al., Differentiation of embryonic stem cells to insulin-secreting structures similar to pancreatic islets. Science, 2001. 292(5520): p. 1389-94.
11. Tanner, S., et al., Accurate Annotation of Peptide Modifications through Unrestrictive Database Search. J Proteome Res, 2008. 7(1): p. 170-81.
12. Tanner, S., et al., Improving gene annotation using peptide mass spectrometry. Genome Res, 2007. 17(2): p. 231-9.

### Example 2

In one embodiment, 11MTAD is a transactivation domain that can be used in a DRP protein of this invention. In one embodiment, a “minimal transactivation domain”, or MTAD, residues 695-781 from human β-catenin (see e.g., Hecht (1999) J. Biol. Chem.

274:18017-18025) can be used. In one embodiment, a Krüppel-associated box (KRAB) domain of KOX1 (residues 1-75) is used, e.g., a Krüppel-associated box (KRAB) domain of KOX1, residues 1 to 75, e.g. as described by Margolin (1994) Proc. Natl. Acad. Sci. USA 91:4509-4513, and Tachikawa (2004) Proc. Natl. Acad. Sci. USA 101:1525-15230, is used as a repression domain.

We replaced the VP16 transactivation domain of Oct-4-D DRP with 11MTAD and tested its activity using luciferase expression system using a transient reporter transfection system in a human embryonic kidney cell line (HEK293) and a mouse embryonic stem cell line. While Oct-4-D DRP with VP16 gave approximately 3-times higher luciferase expression compare to basal activity of the transfected Oct-4-promoter reporter, Oct-4-D DRP with 11MTAD induced approximately 2.5-times higher effect than VP16, resulting in 7.5-fold induction in luciferase activity in HEK293 cells. Similar results were observed in a mouse 129 embryonic stem cell line and in other promoter-reporter systems.

These data demonstrate that 11MTAD can be effectively used in compositions of this invention, e.g., DRP chimeric proteins of the invention, as a universal transactivator with in some applications higher and longer lasting activity than VP16. In alternative embodiments of this invention, for any DRP or ReD (Reprogrammed DRP) of the invention, a VP16 domain can be replaced with 11MTAD.

In alternative embodiments, the following exemplary nucleic acid constructs can be used to practice this invention. Cloning sites, BamH I and Avr II are shown in green and red, respectively; or alternatively, both sites are underlined, where the BamHI site is the underlined “ggatcc” encoding the amino acid residues “GS”, and the AvrII site is the underlined “cctagg” encoding the amino acid residues “PR”. The DRP or ReD's zinc finger domain is in between BamH I and Avr II site. Thus, each sequence indicated has an R9-CPP (cell-penetrating peptide) and NLS (Nuclear Localization Signal; which also can be calls a nuclear localization peptide, or NLP) at upstream of BamH I site and VP16, and a KRAB or an 11MTAD downstream of Avr II site.

An exemplary DRP or ReD protein-encoding sequence of the invention comprises:

Oct4-B DRP with VP16 (mammalian)
(SEQ ID NO: 8)

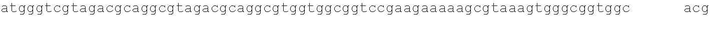

ggtgagaagccgtataaatgtcccgaatgtggtaaaagttttagccgctcgaccgatctgcaaagacatcaacgcacccataccg
gcgaaaaaccatacaaatgtccggagtgcggcaaatctttctcgcgcagcgataacttgcagcagcatcagagaactcacactg
gcgagaagccctacaagtgccccgaatgcgggaagagctttagtcgttctaccaacttacaacgccaccagaggacgcatacc
ggtgagaagccgtataaatgtcccgaatgtggtaaaagttttagcacctcggatcatctgcaaagacatcaacgcacccataccg
gcgaaaaaccatacaaatgtccggagtgcggcaaatctttctcgcgtagcgatcatttgcagcgccatcagagaactcacactgg
cgagaagccctacaagtgccccgaatgcgggaagagctttagtacctctgatcatttacaacgtcaccagaggacgcatacggg

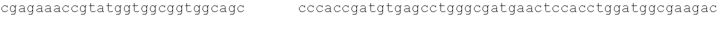

gttgctatggcccatgcagatgctcttgatgactttgatctggacatgcttggagatggtgacagccctggccccggatttacccct
catgatagcgcaccctatggagcactggatatggccgatttcgaatttgaacagatgtttaccgatgcactcggaattgatgaatat
ggcggagctagcgattataaagatgacgatgacaaataa

An exemplary DRP or ReD protein of the invention comprises the amino acid sequence:

(SEQ ID NO: 9)

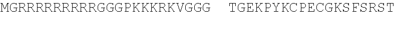

DLQRHQRTHTGEKPYKCPECGKSFSRSDNLQQHQRTHTGEKPYKC
PECGKSFSRSTNLQRHQRTHTGEKPYKCPECGKSFSTSDHLQRHQ
RTHTGEKPYKCPECGKSFSRSDHLQRHQRTHTGEKPYKCPECGKS

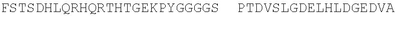

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

Oct4-D DRP with VP16 (mammalian)
(SEQ ID NO: 10)

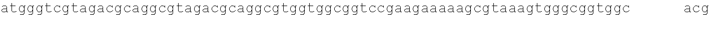

ggtgagaagccgtataaatgtcccgaatgtggtaaaagttttagccgctcgacccatctgcaaagacatcaacgcacccataccg
gcgaaaaaccatacaaatgtccggagtgcggcaaatctttctcgcgcagcgatagtttgcagaggcatcagagaactcacactg
gcgagaagccctacaagtgccccgaatgcgggaagagctttagtacctctgatcatttacaacgtcaccagaggacgcataccg
gtgagaagccgtataaatgtcccgaatgtggtaaaagttttagcagatcggatagcctgcaagaacatcaacgcacccataccgg
cgaaaaaccatacaaatgtccggagtgcggcaaatctttctcgcagagctcgaacttgcagcgccatcagagaactcacactgg
cgagaagccctacaagtgccccgaatgcgggaagagctttagtaggtctgataacttacaaagacaccagaggacgcatacgg

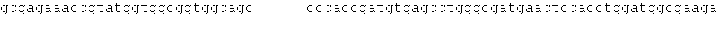

cgttgctatggcccatgcagatgctcttgatgactttgatctggacatgcttggagatggtgacagccctggccccggatttacccc
tcatgatagcgcaccctatggagcactggatatggccgatttcgaatttgaacagatgtttaccgatgcactcggaattgatgaatat
ggcggagctagcgattataaagatgacgatgacaaataa

An exemplary DRP or ReD protein of the invention comprises the amino acid sequence:

(SEQ ID NO: 11)

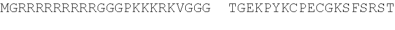

HLQRHQRTHTGEKPYKCPECGKSFSRSDSLQRHQRTHTGEKPYKC
PECGKSFSTSDHLQRHQRTHTGEKPYKCPECGKSFSRSDSLQEHQ
RTHTGEKPYKCPECGKSFSQSSNLQRHQRTHTGEKPYKCPECGKS

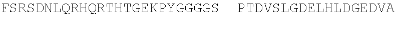

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

VEGF(+500)-DRP with VP16 (mammalian)
(SEQ ID NO: 12)

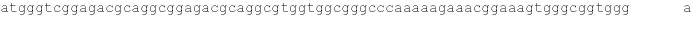

cgggggagaagccgtataaatgccccgaatgtggtaaaagtttttctgaaacaacagcctgcagcgtcaccaacggacccata
ctggcgaaaaaccatacaaatgtcccgagtgcggcaaatctttcagcacctctgatcatttgcagcggcatcaacgcactcacact
ggcgagaagccctacaagtgtccggaatgcgggaagagctttagtgaaagcgatcatctgcaacgtcaccagaggacgcatac
cggtgagaagccgtataaatgccccgaatgtggtaaaagtttttctcgtagcgatcatctgcagcgtcaccaacggacccatactg
gcgaaaaaccatacaaatgtcccgagtgcggcaaatctttcagcacctctgatgatttgcagcgtcatcaacgcactcacactgg
cgagaagccctacaagtgtccggaatgcgggaagagctttagtcgtagcgatcatctgcaacgtcaccagaggacgcataccg

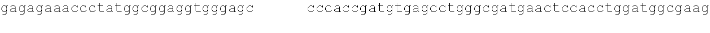

acgttgctatggcccatgcagatgctcttgatgactttgatctggacatgcttggagatggtgacagccctggccccggatttaccc
ctcatgatagcgcaccctatggagcactggatatggccgatttcgaatttgaacagatgtttaccgatgcactcggaattgatgaat
atggcggagctagcgattataaagatgacgatgacaaataa

An exemplary DRP or ReD protein of the invention comprises the amino acid sequence:

(SEQ ID NO: 13)

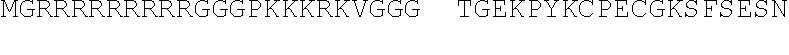

SLQRHQRTHTGEKPYKCPECGKSFSTSDHLQRHQRTHTGEKPYKC
PECGKSFSESDHLQRHQRTHTGEKPYKCPECGKSFSRSDHLQRHQ
RTHTGEKPYKCPECGKSFSTSDDLQRHQRTHTGEKPYKCPECGKS

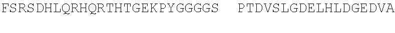

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

VEGF(−500)-DRP with VP16 (mammalian)
(SEQ ID NO: 14)

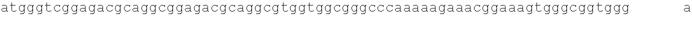

cgggggagaagccgtataaatgccccgaatgtggtaaaagtttttctaccagcgatcatctgcagactcaccaacggaccatac
tggcgaaaaaccatacaaatgtcccgagtgcggcaaatctttcagcaggtctaataacttgcagcggcatcaacgcactcacact
ggcgagaagccctacaagtgtccggaatgcgggaagagctttagtaccagcgatcatctgcaacagcaccagaggacgcata
ccggtgagaagccgtataaatgccccgaatgtggtaaaagtttttctcgtagcaccagtctgcagcgtcaccaacggacccatac
tggcgaaaaaccatacaaatgtcccgagtgcggcaaatctttcagcaggtctgatcacttgcagacccatcaacgcactcacact
ggcgagaagccctacaagtgtccggaatgcgggaagagctttagtcgtagcaactccctgcaacgtcaccagaggacgcatac

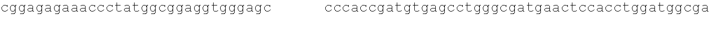

agacgttgctatggcccatgcagatgctcttgatgactttgatctggacatgcttggagatggtgacagccctggccccggatttac
ccctcatgatagcgcaccctatggagcactggatatggccgatttcgaatttgaacagatgtttaccgatgcactcggaattgatga
atatggcggagctagcgattataaagatgacgatgacaaataa

An exemplary DRP or ReD protein of the invention comprises the amino acid sequence:

(SEQ ID NO: 15)

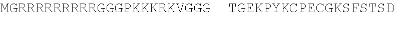

HLQTHQRTHTGEKPYKCPECGKSFSRSNNLQRHQRTHTGEKPYKC
PECGKSFSTSDHLQQHQRTHTGEKPYKCPECGKSFSRSTSLQRHQ
RTHTGEKPYKCPECGKSFSRSDHLQTHQRTHTGEKPYKCPECGKS

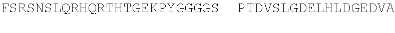

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

VEGF(+500)-DRP with VP16 (E. coli)
(SEQ ID NO: 16)

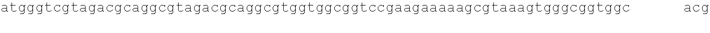

ggtgagaagccgtataaatgtcccgaatgtggtaaaagttttagcgaatcgaacagcctgcaaaggcatcaacgcacccatacc
ggcgaaaaaccatacaaatgtccggagtgcggcaaatctttctcgaccagcgatcatttgcagagacatcagagaactcacact
ggcgagaagccctacaagtgccccgaatgcgggaagagctttagtgaatctgatcacttacaacgccaccagaggacgcatac
cggtgagaagccgtataaatgtcccgaatgtggtaaaagttttagcaggtcggatcatctgcaacgtcatcaacgcacccatacc
ggcgaaaaaccatacaaatgtccggagtgcggcaaatctttctcgaccagcgatgacttgcagagacatcagagaactcacact
ggcgagaagccctacaagtgccccgaatgcgggaagagctttagtagatctgatcatttacaaaggcaccagaggacgcatac

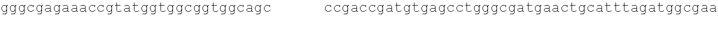

gatgtggcgatggcccatgcggatgccctagacgattttgacctggatatgttaggcgatggtgacagccccggtccgggtttta
ccccgcatgatagcgcaccgtatggtgcgctagatatggcggatttcgaatttgaacagatgtttaccgatgcgctgggtattgatg
aatatggcggtgctagcgattataaagatgacgatgacaaataa

An exemplary DRP or ReD protein of the invention comprises the amino acid sequence:

(SEQ ID NO: 17)

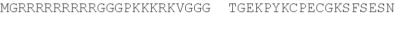

SLQRHQRTHTGEKPYKCPECGKSFSTSDHLQRHQRTHTGEKPYKC
PECGKSFSESDHLQRHQRTHTGEKPYKCPECGKSFSRSDHLQRHQ
RTHTGEKPYKCPECGKSFSTSDDLQRHQRTHTGEKPYKCPECGKS

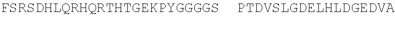

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

VEGF(−500)-DRP with VP16 (E. coli)
(SEQ ID NO: 18)

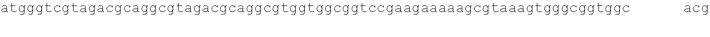

ggtgagaagccgtataaatgtcccgaatgtggtaaaagttttagcacctcggatcatctgcaaacccatcaacgcacccataccg
gcgaaaaaccatacaaatgtccggagtgcggcaaatctttctcgcgcagcaataacttgcagagacatcagagaactcacactg
gcgagaagccctacaagtgccccgaatgcgggaagagctttagtacctctgaccacttacaacagcaccagaggacgcatacc
ggtgagaagccgtataaatgtcccgaatgtggtaaaagttttagcaggtcgaccagcctgcaacgccatcaacgcacccatacc
ggcgaaaaaccatacaaatgtccggagtgcggcaaatctttctcgcgcagcgatcatttgcagacccatcagagaactcacact
ggcgagaagccctacaagtgccccgaatgcgggaagagctttagtagatctaacagcttacaaaggcaccagaggacgcata

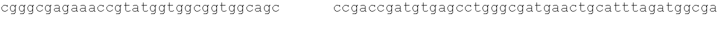

agatgtggcgatggcccatgcggatgccctagacgattttgacctggatatgttaggcgatggtgacagccccggtccgggtttt
accccgcatgatagcgcaccgtatggtgcgctagatatggcggatttcgaatttgaacagatgtttaccgatgcgctgggtattgat
gaatatggcggtgctagcgattataaagatgacgatgacaaataa

An exemplary DRP or ReD protein of the invention comprises the amino acid sequence:

(SEQ ID NO: 19)

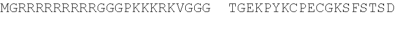

HLQTHQRTHTGEKPYKCPECGKSFSRSNNLQRHQRTHTGEKPYKC
PECGKSFSTSDHLQQHQRTHTGEKPYKCPECGKSFSRSTSLQRHQ
RTHTGEKPYKCPECGKSFSRSDHLQTHQRTHTGEKPYKCPECGKS

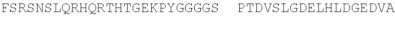

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

VEGF(+500)-DRP with 11MTAD (mammalian)
(SEQ ID NO: 20)

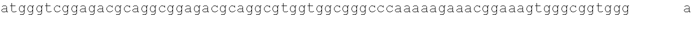

cgggggagaagccgtataaatgccccgaatgtggtaaaagtttttctgaaagcaacagcctgcagcgtcaccaacggacccata
ctggcgaaaaaccatacaaatgtcccgagtgcggcaaatctttcagcacctctgatcatttgcagcggcatcaacgcactcacact
ggcgagaagcctacaagtgtccggaatgcgggaagagctttagtgaaagcgatcatctgcaacgtcaccagaggacgcatac
cggtgagaagccgtataaatgccccgaatgtggtaaaagtttttctcgtagcgatcatctgcagcgtcaccaacggacccatactg
gcgaaaaaccatacaaatgtcccgagtgcggcaaatctttcagcacctctgatgatttgcagcgtcatcaacgcactcacactgg
cgagaagccctacaagtgtccggaatgcgggaagagctttagtcgtagcgatcatctgcaacgtcaccagaggacgcataccg

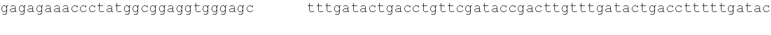

tgacctgttcgataccgacttgtttgatactgacctttttgatactgacctgttcgataccgacttgtttgatactgacctttttgatactga
cctgttcgataccgacttggtggcggtgctagcgattataaagatgacgatgacaaataa

An exemplary DRP or ReD protein of the invention comprises the amino acid sequence:

(SEQ ID NO: 21)

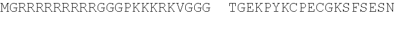

SLQRHQRTHTGEKPYKCPECGKSFSTSDHLQRHQRTHTGEKPYKC
PECGKSFSESDHLQRHQRTHTGEKPYKCPECGKSFSRSDHLQRHQ
RTHTGEKPYKCPECGKSFSTSDDLQRHQRTHTGEKPYKCPECGKS

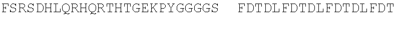

DLFDTDLFDTDLFDTDLFDTDLFDTDLFDTDLFDTDLGGGASDYK
DDDDK*

An exemplary DRP or ReD protein-encoding sequence of the invention comprises:

VEGF(−500)-DRP with MTAD (mammalian)
(SEQ ID NO: 22)

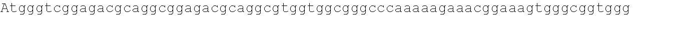

acgggggagaagccgtataaatgccccgaatgtggtaaaagtttttctaccagcgatcatctgcagactcaccaacggacccata
ctggcgaaaaaccatacaaatgtcccgagtgcggcaaatctttcagcaggtctaataacttgcagcggcatcaacgcactcacac
tggcgagaagccctacaagtgtccggaatgcgggaagagctttagtaccagcgatcatctgcaacagcaccagaggacgcata
ccggtgagaagccgtataaatgccccgaatgtggtaaaagtttttctcgtagcaccagtctgcagcgtcaccaacggaccatac
tggcgaaaaaccatacaaatgtcccgagtgcggcaaatctttcagcaggtctgatcacttgcagacccatcaacgcactcacact
ggcgagaagccctacaagtgtccggaatgcgggaagagctttagtcgtagcaactccctgcaacgtcaccagaggacgcatac

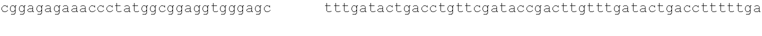

tactgacctgttcgataccgacttgtttgatactgacctttttgatactgacctgttcgataccgacttgtttgatactgacctttttgatac
tgacctgttcgataccgacttggtggcggtgctagcgattataaagatgacgatgacaaataa

An exemplary DRP or ReD protein of the invention comprises the amino acid sequence:

(SEQ ID NO: 23)

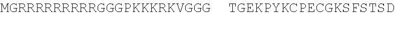

HLQTHQRTHTGEKPYKCPECGKSFSRSNNLQRHQRTHTGEKPYKC
PECGKSFSTSDHLQQHQRTHTGEKPYKCPECGKSFSRSTSLQRHQ
RTHTGEKPYKCPECGKSFSRSDHLQTHQRTHTGEKPYKCPECGKS

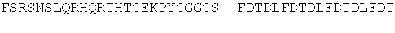

DLFDTDLFDTDLFDTDLFDTDLFDTDLFDTDLFDTDLGGGASDYK
DDDDK*

An exemplary DRP or ReD protein of the invention comprises the amino acid sequence:
An exemplary DRP or ReD protein-encoding sequence of the invention comprises:

VEGF(+500)-DRP with KRAB (mammalian)
(SEQ ID NO: 24)

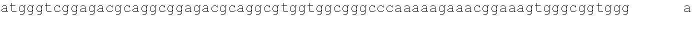

cgggggagaagccgtataaatgccccgaatgtggtaaaagtttttctgaaagcaacagcctgcagcgtcaccaacggacccata
ctggcgaaaaaccatacaaatgtcccgagtgcggcaaatctttcagcacctctgatcatttgcagcggcatcaacgcactacact
ggcgagaagccctacaagtgtccggaatgcgggaagagctttagtgaaagcgatcatctgcaacgtcaccagaggacgatac
cggtgagaagccgtataaatgccccgaatgtggtaaaagtttttctcgtagcgatcatctgcagcgtcaccaacggacccatactg
gcgaaaaaccatacaaatgtcccgagtgcggcaaatctttcagcacctctgatgatttgcagcgtcatcaacgcactcacactgg
cgagaagccctacaagtgtccggaatgcgggaagagctttagtcgtagcgatcatctgcaacgtcaccagaggacgcataccg

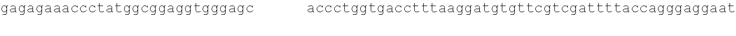

ggaaactgttggacaccgcccaacagattgtctatagaaatgtgatgctggagaactataagaatctggtgagcttgggctatcag
ctgaccaaacccgacgtgattctgagactcgaaaagggcgaggaaccctggctggtgggcggagctagcgattataaagatga
cgatgacaaataa

An exemplary DRP or ReD protein of the invention comprises the amino acid sequence:

(SEQ ID NO: 25)

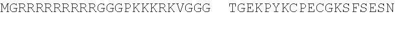

SLQRHQRTHTGEKPYKCPECGKSFSTSDHLQRHQRTHTGEKPYKC
PECGKSFSESDHLQRHQRTHTGEKPYKCPECGKSFSRSDHLQRHQ
RTHTGEKPYKCPECGKSFSTSDDLQRHQRTHTGEKPYKCPECGKS

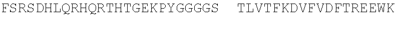

LLDTAQQIVYRNVMLENYKNLVSLGYQLTKPDVILRLEKGEEPWL
VGGASDYKDDDDK*

An exemplary DRP or ReD protein-encoding sequence of the invention comprises:

VEGF(−500)-DRP with KRAB (mammalian)
(SEQ ID NO: 26)

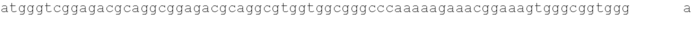

cgggggagaagccgtataaatgccccgaatgtggtaaaagtttttctaccagcgatcatctgcagactcaccaacggaccatac
tggcgaaaaaccatacaaatgtcccgagtgcggcaaatctttcagcaggtctaataacttgcagcggcatcaacgcactcacact
ggcgagaagccctacaagtgtccggaatgcgggaagagctttagtaccagcgatcatctgcaacagcaccagaggacgcata
ccggtgagaagccgtataaatgccccgaatgtggtaaaagtttttctcgtagcaccagtctgcagcgtcaccaacggacccatac
tggcgaaaaaccatacaaatgtcccgagtgcggcaaatctttcagcaggtctgatcacttgcagacccatcaacgcactcacact
ggcgagaagccctacaagtgtccggaatgcgggaagagctttagtcgtagcaactccctgcaacgtcaccagaggacgcatac

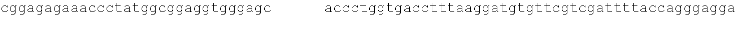

atggaaactgttggacaccgcccaacagattgtctatagaaatgtgatgctggagaactataagaatctggtgagcttgggctatc
agctgaccaaacccgacgtgattctgagactcgaaaagggcgaggaccctggctggtgggcggagctagcgattataaagat
gacgatgacaaataa

An exemplary DRP or ReD protein of the invention comprises the amino acid sequence:

(SEQ ID NO: 27)

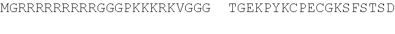

HLQTHQRTHTGEKPYKCPECGKSFSRSNNLQRHQRTHTGEKPYKC
PECGKSFSTSDHLQQHQRTHTGEKPYKCPECGKSFSRSTSLQRHQ
RTHTGEKPYKCPECGKSFSRSDHLQTHQRTHTGEKPYKCPECGKS

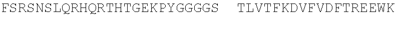

LLDTAQQIVYRNVMLENYKNLVSLGYQLTKPDVILRLEKGEEPWL
VGGASDYKDDDDK*

An exemplary DRP or ReD protein-encoding sequence of the invention comprises:

Nanog_1-DRP with VP 16 (mammalian)-target sequence: 731-gggggtgggtagggtagga-749
(SEQ ID NO: 28)

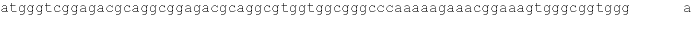

cgggtgagaagccgtataaatgtcccgaatgtggtaaaagttttagcaggtcgacccatctgcaacagcatcaacgcaccatac
cggcgaaaaaccatacaaatgtccggagtgcggcaaatctttctcgacgagcacccacttgcagaggcatcagagaactcaca
ctggcgagaagccctacaagtgccccgaatgcgggaagagctttagtcgttctgataatttacaaacccaccagaggacgcata
ccggtgagaagccgtataaatgtcccgaatgtggtaaaagttttagccgttcgaatcatctgcaaagacatcaacgcaccatacc
ggcgaaaaaccatacaaatgtccggagtgcggcaaatctttctcgaccagcgaccacttgcagagacatcagagaactcacact
ggcgagaagccctacaagtgccccgaatgcgggaagagctttagtagatctgatcatttacaacgtcaccagaggacgcatacg

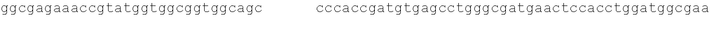

gacgttgctatggcccatgcagatgctcttgatgactttgatctggacatgcttggagatggtgacagccctggccccggatttacc
cctcatgatagcgcaccctatggagcactggatatggccgatttcgaatttgaacagatgtttaccgatgcactcggaattgatgaa
tatggcggagctagcgattataaagatgacgatgacaaataa

An exemplary DRP or ReD protein of the invention comprises the amino acid sequence:

(SEQ ID NO: 29)

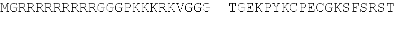

HLQQHQRTHTGEKPYKCPECGKSFSTSTHLQRHQRTHTGEKPYKC
PECGKSFSRSDNLQTHQRTHTGEKPYKCPECGKSFSRSNHLQRHQ
RTHTGEKPYKCPECGKSFSTSDHLQRHQRTHTGEKPYKCPECGKS

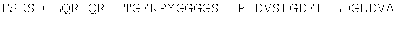

MAHADALDDFDLDMLGDGDSPGPGFTPHDSAPYGALDMADFEFEQ
MFTDALGIDEYGGASDYKDDDDK*

An exemplary DRP or ReD protein-encoding sequence of the invention comprises:

Nanog_2-DRP with VP16 (mammalian)-target sequence: 548-ttttgggggggggggatgt-566
(SEQ ID NO: 30)

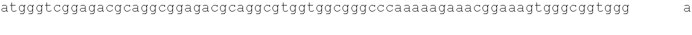

cgggtgagaagccgtataaatgtcccgaatgtggtaaaagttttagcagatcgaattctctgcaacagcatcaacgcacccatacc
ggcgaaaaaccatacaaatgtccggagtgcggcaaatctttctcgagaagcacccacttgcagaggcatcagagaactcacact
ggcgagaagcctacaagtgccccgaatgcgggaagactttagtaggtctgatcacttacaaaggcaccagaggacgcatac
cggtgagaagccgtataaatgtcccgaatgtggtaaaagttttagccgttcggaccacctgcaaagacatcaacgcacccatacc
ggcgaaaaaccatacaaatgtccggagtgcggcaaatctttctcgcgtagcgatcatttgcagacgcatcagagaactcacactg
gcgagaagccctacaagtgccccgaatgcgggaagagctttagtacctctaactctttacaaacgcaccagaggacgcatacg

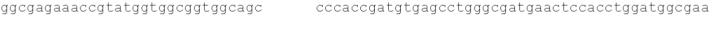

gacgttgctatggcccatgcagatgctcttgatgactttgatctggacatgcttggagatggtgacagccctggccccggatttacc
cctcatgatagcgcaccctatggagcactggatatggccgatttcgaatttgaacagatgtttaccgatgcactcggaattgatgaa
tatggcggagctagcgattataaagatgacgatgacaaataa

An exemplary DRP or ReD protein of the invention comprises the amino acid sequence:

(SEQ ID NO: 31)

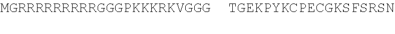

SLQQHQRTHTGEKPYKCPECGKSFSRSTHLQRHQRTHTGEKPYKC
PECGKSFSRSDHLQRHQRTHTGEKPYKCPECGKSFSRSDHLQRHQ
RTHTGEKPYKCPECGKSFSRSDHLQTHQRTHTGEKPYKCPECGKS

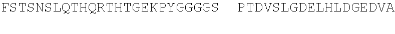

MAHADALDDFDLDMLGDGDSPGPGFTPHDSAPYGALDMADFEFEQ
MFTDALGIDEYGGASDYKDDDDK*

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a plurality of Designed Regulatory Proteins (DRPs) or Reprogramming DRP proteins (ReDs), or provides a plurality of DRPs or ReDs, or the composition comprises one or at least one DRP or ReD chimeric protein that can bind to and activate the transcription of each member of the combination of genes set forth below, wherein
(a) each DRP or ReD is a chimeric protein comprising:
(1) at least one zinc finger DNA binding peptide domain specific for (capable of specifically binding to) a promoter or a transcriptional regulatory region of a gene;
(2) at least one nuclear localization peptide (NLP) domain;
(3) at least one cell-penetrating peptide (CPP); and,
(4) a transcription activation peptide domain and/or a transcription repression peptide domain; and
(b) at least one transcription activation peptide domain of each DRP or ReD chimeric protein can bind to and activate the transcription of at least one of the following genes, and the composition comprises at least one DRP or ReD chimeric protein that can bind to and activate the transcription of each member of the combination of genes selected from the group consisting of:
(1) a combination of genes consisting of an Oct4, a Sox2, a Klf4, a c-Myc, a Lin28 and a Nanog gene;
(2) a combination of genes consisting of any five of the following genes: Oct4, Sox2, Klf4, c-Myc, Lin28, Nanog;
(3) a combination of genes consisting of any four of the following genes: Oct4, Sox2, Klf4, c-Myc, Lin28, Nanog;
(4) a combination of genes consisting of any three of the following genes: Oct4, Sox2, Klf4, c-Myc, Lin28, Nanog;
(5) a combination of genes consisting of any two of the following genes: Oct4, Sox2, Klf4, c-Myc, Lin28, Nanog;
(6) a combination of genes consisting of an Oct4 or a Sox2 gene, and a Klf4 or a Nanog gene;
(7) a combination of genes consisting of an Oct4, a Sox2, and a Klf4 or a Nanog gene; or
(8) a combination of genes consisting of an Oct4 gene, or a Sox2 gene, or a Klf4 or a Nanog gene.

2. The composition of claim 1, wherein the at least one DRP or ReD chimeric protein comprises a recombinant protein, a synthetic protein, a peptidomimetic, a non-natural peptide, or a combination thereof.

3. The composition of claim 1, wherein the chimeric protein comprises multiple copies of the zinc finger DNA binding peptide domain, the NLP, the CPP and/or the transcription activation peptide.

4. The composition of claim 1, wherein a different DRP or ReD chimeric protein binds to and activates the transcription of each gene in the combination, or one of the DRP or ReD chimeric proteins can bind to and activate the transcription of two different genes in the combination, or one of the DRP chimeric proteins can bind to and activate the transcription of three or more different genes in the combination.

5. The composition of claim 1, wherein the combination of genes further comprises at least one member of the Myc family of transcription factors.

6. The composition of claim 5, wherein the at least one member of the Myc family of transcription factors is a N-Myc, a L-Myc or a c-Myc gene.

7. The composition of claim 1, wherein the least one DRP or ReD chimeric protein has or further comprises at least one transcription repression peptide domain that represses the transcription of a Pax5 message (mRNA, transcript).

8. The composition of claim 1, wherein the at least one DRP or ReD chimeric protein has or further comprises at least one transcription repression peptide domain that represses the transcription of a (zinc finger transcription factor) GATA6 gene, or the repression peptide domain comprises a Krüppel-associated box (KRAB) domain of KOX1, or the repression peptide domain comprises an SRDX domain from Arabidopsis thaliana SUPERMAN protein.

9. The composition of claim 1, wherein the at least one zinc finger binding peptide domain comprises (1) a zinc-finger of the C₂H₂ class; (2) a zinc-finger of the C₄ class; or (3) a zinc-finger of C₆ class.

10. The composition of claim 1, wherein the at least one zinc finger binding peptide domain comprises the consensus sequence Cys-X₂₋₄-Cys-X₃-Phe-X₅-Leu-X₂-His-X3-His (SEQ ID NO:1).

11. The composition of claim 1, wherein the at least one nuclear localization peptide (NLP) domain comprises
(1) an NLP sequence of a large T antigen of the simian virus 40 (SV-40), or PKKKRKV (SEQ ID NO:2);
(2) a consensus sequence fitting B₄ (SEQ ID NO:3), P(B₃X) (SEQ ID NO:4), PXX(B₃X) (SEQ ID NO:5), B₃(H/P) (SEQ ID NO:6), where B is a basic amino acid, P is proline, H is histidine, X is any amino acid and letters in parentheses can be in any order;
(3) a bipartite NLP comprising two short stretches of basic amino acids separated by a non-conserved sequence; or
(4) a cellular nucleoplasmin protein KRPAATKKAGQAKKKK (SEQ ID NO:7).

12. The composition of claim 1, wherein the at least one cell-penetrating peptide (CPP) comprises
(1) a plurality of polycationic amino acid residues;
(2) a plurality of arginine amino acid residues; or
(3) a TAT protein (Trans-acting Activator of Transcription) of a Human Immunodeficiency Virus (HIV-1).

13. The composition of claim 1, wherein
(1) the at least one transcription activation peptide domain is at least approximately 25% hydrophobic and is linked to the at least one zinc finger binding peptide in a manner that does not interfere with the promoter or a transcriptional regulatory binding activity of the zinc finger DNA binding peptide, and the transcription activation peptide is both necessary and sufficient to activate transcription of the gene; and/or
(2) the transcription activation peptide domain is between about 5 to 25 amino acids in length, or is between about 6 to 20 amino acids in length, or is about 5, 6, 7, 8, 9, 10, 11, 11, 12, 13, 14 or 15 amino acids in length.

14. The composition of claim 1, wherein the at least one transcription activation peptide domain comprises a herpes simplex virus (HSV) VP-16 activation peptide domain or a peptide derived from the C-terminal transcription activation domain of β-catenin (FDTDL).

15. The composition of claim 1, wherein at least one, or all, of the DRP or ReD chimeric proteins further comprises, or is attached to, a lipid or a polyethylene glycol (PEG) moiety.

16. The composition of claim 1, wherein at least one, or all, of the DRP or ReD chimeric proteins further comprises, or is attached to, an epitope peptide tag or a detectable composition or moiety.

17. The composition of claim 16, wherein the composition comprises a phosphoprotein, a fluorescent molecule, a fluorescent tagged protein, a radiolabel or a radiolabeled protein.

18. The composition of claim 1, wherein the composition further comprises a small molecule, a hormone or a cytokine that has a de-differentiation (re-programming) effect on the mammalian cell.

19. The composition of claim 18, wherein the cytokine comprises a transforming growth factor-beta (TGF-beta).

20. The composition of claim 1, wherein the composition further comprises a large T antigen of the simian virus 40 (SV-40), or any protein or peptide that inhibits the activity of tumor suppressor gene retinoblastoma-1 (RB1) and/or p53 tumor suppressor gene (TP53).

21. The composition of claim 1, wherein the composition further comprises a protein or peptide comprising or consisting of a catalytic subunit of TERT.

22. The composition of claim 21, wherein the catalytic subunit of TERT is hTERT.

23. The composition of claim 1, wherein the composition further comprises a histone deacetylase inhibitor, or further comprises a histone deacetylase inhibitor comprising a valproic acid (VPA).

24. The composition of claim 23, wherein a Designed Regulatory Protein (DRP) or a Reprogramming DRP protein (ReD):
(a) is encoded by a nucleic acid comprising SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, or SEQ ID NO:30;
(b) has an amino acid sequence comprising SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.

25. A liquid, gel, hydrogel, powder or aqueous formulation comprising the composition of any of claims 1 to 24.

26. A vesicle, liposome, nanoparticle or nanolipid particle (NLP) comprising the composition of any of claims 1 to 24, or the liquid, gel, hydrogel, powder or aqueous formulation of claim 25.

27. An isolated or cultured cell comprising (or having contained therein) the composition of any of claims 1 to 24, or the liquid, gel, hydrogel, powder or aqueous formulation of claim 25, or the vesicle, liposome, nanoparticle or nanolipid particle (NLP) of claim 26.

28. The isolated or cultured cell of claim 27, wherein the cell is a mammalian cell.

29. The isolated or cultured cell of claim 28, wherein the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

30. A pharmaceutical or sterile formulation comprising the mammalian cell of claim 28 or claim 29.

31. A product of manufacture comprising an isolated or cultured cell of claim 27.

32. An artificial organ or implant comprising an isolated or cultured cell of claim 27.

33. The artificial organ or implant of claim 32, comprising or formed as an artificial tissue or organ, or an orthopedic implant, an ocular implant, a dental implant, an auricular implant, or a heart valve bio-prosthesis, or a bioactive wound dressing.

34. An in vitro or ex vivo method for de-differentiating or re-programming a mammalian cell comprising:
(a) (i) providing
(1) the composition of claim 1,
(2) the liquid or aqueous formulation of claim 2,
(3) the vesicle, liposome, nanoparticle or nanolipid particle of claim 3, or
(4) a plurality of Designed Regulatory Proteins (DRPs) or Reprogramming DRP protein (ReD), wherein each DRP or ReD is a chimeric protein comprising: (I) at least one zinc finger DNA binding peptide domain specific for (capable of specifically binding to) a promoter or a transcriptional regulatory region of a gene, (II) at least one nuclear localization peptide (NLP) domain, (III) at least one cell-penetrating peptide (CPP), and (IV) a transcription activation peptide domain and/or a transcription repression peptide domain;
wherein the at least one transcription activation peptide domain of each DRP or ReD chimeric protein can bind to and activate the transcription of at least one of the following genes, and the plurality comprises at least one DRP or ReD chimeric protein that can bind to and activate the transcription of each member the combination of genes selected from the group consisting of:
(1) a combination of genes consisting of an Oct4, a Sox2, a Klf4, a c-Myc, a Lin28 and a Nanog gene;
(2) a combination of genes consisting of a Sox2, a Klf4, a c-Myc, a Lin28 and a Nanog gene;
(3) a combination of genes consisting of an Oct4, a Klf4, a c-Myc, a Lin28 and a Nanog gene;
(4) a combination of genes consisting of an Oct4, a Sox2, a c-Myc, a Lin28 and a Nanog gene;
(5) a combination of genes consisting of an Oct4, a Sox2, a Klf4, a Lin28 and a Nanog gene;
(6) a combination of genes consisting of an Oct4, a Sox2, a Klf4, a c-Myc and a Nanog gene;
(7) a combination of genes consisting of an Oct4, a Sox2, a Klf4, a c-Myc and a Lin28 gene;
(8) a combination of genes consisting of any four of the following genes: Oct4, Sox2, Klf4, c-Myc, Lin28; Nanog;
(9) a combination of genes consisting of any three of the following genes: Oct4, Sox2, Klf4, c-Myc, Lin28; Nanog;
(10) a combination of genes consisting of any two of the following genes: Oct4, Sox2, Klf4, c-Myc, Lin28; Nanog;
(11) a combination of genes consisting of an Oct4 or a Sox2 gene, and a Klf4 or a Nanog gene
(12) a combination of genes consisting of an Oct4, a Sox2, and a Klf4 or a Nanog gene;
(13) a combination of genes consisting of an Oct4 gene, or a Sox2 gene, or a Klf4 or a Nanog gene; or
(14) the combination of genes of any of (1) to (13), wherein the combination of genes further comprises at least one member of the Myc family of transcription factors, or an N-Myc, L-Myc or c-Myc gene;
(ii) providing a mammalian cell more differentiated than a pluripotent phenotype; and
(iii) contacting in vitro or ex vivo the composition, or the liquid or aqueous formulation, or the vesicle, liposome, nanoparticle or nanolipid particle, or the plurality of DRPs or ReDs, with the mammalian cell in an amount effective to cause the de-differentiation or re-programming of the mammalian cell.

35. The method of claim 34, wherein the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

36. The method of claim 34, wherein the in vitro or ex vivo contacting of the mammalian cell with the composition, or the liquid or aqueous formulation, or the vesicle, liposome, nanoparticle or nanolipid particle, or the plurality of DRPs or ReDs, is in an aqueous cell culture environment, or the in vitro or ex vivo contacting is on mammalian cells embedded in a gel, or the in vitro or ex vivo contacting is on a mammalian cell that is adherent on (to) a plate or a fixed or gel structure.

37. The method of claim 34, wherein the mammalian cell is contacted with the composition, or the liquid or aqueous formulation, or the vesicle, liposome, nanoparticle or nanolipid particle, or the plurality of DRPs or ReDs, in an amount effective to cause the de-differentiation or re-programming of the mammalian cell to a pluripotent cell.

38. The method of claim 34, wherein the mammalian cell is contacted with the composition, or the liquid or aqueous formulation, or the vesicle, liposome, nanoparticle or nanolipid particle, or the plurality of DRPs or ReDs, in an amount effective to cause the de-differentiation or re-programming of the mammalian cell to a pluripotent cell, a multipotent stem cell, a unipotent stem cell or a totipotent stem cell.

39. The method of claim 34, wherein the mammalian cell of step (a)(ii), before de-differentiation or re-programming, is an endodermal cell, a mesodermal cell or an ectodermal cell.

40. The method of claim 34, wherein the mammalian cell of step (a)(ii), before de-differentiation or re-programming, is an adult stem cell, an embryonic stem cell, a somatic stem cell, an adipose-derived stem cell (ASC), a stem cell derived from an epithelial cell or tissue, a hematopoietic stem cell, a mammary stem cell, a mesenchymal stem cell, a neural stem cell, an olfactory adult stem cell, a spermatogonial progenitor cell, a dental pulp-derived stem cell, or a cancer stem cell.

41. The method of claim 34, wherein the mammalian cell of step (a)(ii), before de-differentiation or re-programming, is an adult somatic cell or an adult germ cell.

42. The method of claim 41, wherein the adult somatic cell, before de-differentiation or re-programming, is a hematopoietic cell, a lymphocyte, a macrophage, a T cell, a B cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an embryoid body cell, an ovary cell, a testis cell, an adipose tissue (fat) cell, or a cancer cell.

43. The method of claim 34, wherein each chimeric protein in the cell culture aqueous environment has a concentration of at least between about 5 to 1000 μgm per ml, or between about 10 to 500 μgm per ml, or between about 50 to 100 μgm per ml; or the mammalian cells are contacted with an aqueous solution or culture media wherein each chimeric protein has a concentration in the aqueous solution or culture media of at least between about 5 to 1000 μgm per ml, or between about 10 to 500 μgm per ml, or between about 50 to 100 μgm per ml.

44. The method of any of claims 34 to 43, wherein the mammalian cell is cultured for between about one to 24 hours, or between about one to two days.

45. The method of claim 44, wherein the mammalian cell is cultured for between about one to 10 days after the in vitro or ex vivo contacting of step (iii).

46. The method of any of claims 34 to 45, wherein the mammalian cell is cultured before, during and/or after the in vitro or ex vivo contacting of step (iii).

47. The method of claim 34, wherein the mammalian cell is also contacted with a cytokine that has a de-differentiation (re-programming) effect on the mammalian cell.

48. The method of claim 46, wherein the cytokine comprises a transforming growth factor-beta (TGF-beta), interleukin-18 (IL-18, or interferon-γ-inducing factor), adipose complement-related protein or interferon-γ.

49. The method of claim 34, wherein the mammalian cell is also contacted with a large T antigen of the simian virus 40 (SV-40), or any protein or peptide or nucleic acid that inhibits the activity of a tumor suppressor gene retinoblastoma-1 (RB1) and/or a p53 tumor suppressor gene (TP53), and the contacting is before, during or after the contacting step of (a)(iii).

50. The method of claim 34, wherein the mammalian cell is also contacted with a protein or peptide comprising or consisting of a catalytic subunit of TERT, or nucleic acid that encodes a catalytic subunit of TERT, and the contacting is before, during or after the contacting step of (a)(iii).

51. The method of claim 50, wherein the catalytic subunit of TERT is hTERT.

52. The method of claim 34, wherein the method further comprises the deletion or inhibition of a gene and/or transcript (mRNA, message) encoding one or more of a set of nucleic acid and/or protein transcription factors responsible for maintaining a differentiated phenotype of the mammalian cell, and/or inhibition of a protein transcription factor responsible for maintaining a differentiated phenotype of the mammalian cell.

53. The method of claim 52, wherein the deletion or inhibition of a gene and/or transcript (mRNA, message) is by expression of or administration of a nucleic acid or protein that is inhibitory to the activity and/or expression of the gene, transcript and/or protein transcription factor.

54. The method of claim 53, wherein the nucleic acid that is inhibitory to the gene and/or transcript comprises an miRNA, an siRNA, a ribozyme and/or an antisense nucleic acid, or the protein that is inhibitory to the activity and/or expression of the gene, transcript and/or protein transcription factor comprises an antibody that specifically binds to the protein transcription factor.

55. The method of claim 34, wherein the one or more of the transcription factors inhibited is Pax5, or the method further comprises inhibiting or knocking out the expression of a gene and/or transcript encoding Pax5.

56. The method of claim 34, wherein the method further comprises addition before, during or after the contacting step of (a)(iii) of a histone deacetylase inhibitor.

57. The method of claim 34, wherein the histone deacetylase inhibitor comprises a valproic acid (VPA).

58. The method of claim 34, further comprising identifying and/or isolating the de-differentiated or re-programmed cell by using an antibody that specifically binds to a polypeptide cell surface marker present in the de-differentiated or re-programmed cell and not the cell before de-differentiating or re-programming.

59. The method of claim 58, wherein the polypeptide cell surface marker present in the de-differentiated or re-programmed cell and not the cell before de-differentiating or re-programming is
(1) CXCR4, CD10, CD13, CD41a (gpIIbIIIa), CD34, CD56, CD90, CD110, CD117, CD123, CD133, CD135, CD277 and/or CD318;
(2) CD10, CD13, CD56, and MHC Class-I cell surface antigens;
(3) the method of (b)(1) or (b)(2), wherein the cells are also negative for
(1) CD3, CD5, CD7, CD11b, CD14, CD15, CD16, CD19, CD25, CD45, and/or CD65 markers, or
(2) CD3, CD4, CD8, CD11c, CD33, CD36, CD38, CD45, CD117, Glycophorin-A and/or HLA DR-II.

60. The method of claim 58 or claim 59, wherein the cell is identified and/or isolated by positive or negative selection using the antibody.

61. The method of claim 60, wherein the identifying and/or isolating the de-differentiated (re-programmed) cell by negative selection of cells still expressing a differentiated cell marker.

62. The method of claim 60, wherein the cell is identified and/or isolated by fluorescent activated cell sorting (FACS) or affinity column chromatography.

63. The method of claim 60, wherein the cell is identified and/or isolated by identification and/or isolation of plasma membrane proteins by mass spectography or chromatography.

64. The method of claim 60, wherein the identifying and/or isolating the de-differentiated (re-programmed) cell by determining the presence or absence of a message (mRNA, transcript) determinative of an undifferentiated cell phenotype.

65. The method of claim 64, wherein the message (mRNA, transcript) determinative of an undifferentiated cell phenotype is a message for Oct4, a Sox2, a Klf4, a c-Myc, a Lin28 and a Nanog gene.

66. The method of any of claims 34 to 65, further comprising implanting the de-differentiated or re-programmed mammalian cell in a vessel, tissue or organ.

67. The method of claim 66, wherein the de-differentiated or re-programmed mammalian cell is implanted in the vessel, tissue or organ ex vivo or in vivo.

68. The method of any of claims 34 to 67, further comprising implanting the de-differentiated or re-programmed mammalian cell in an individual in need thereof.

69. A de-differentiated or re-programmed cell made by practicing the method of any of claims 34 to 68, wherein the de-differentiated or re-programmed cell is a mammalian cell.

70. The de-differentiated or re-programmed cell of claim 68, wherein the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

71. A kit comprising (i) (a) the composition comprising the plurality of Designed Regulatory Proteins (DRPs) or ReDs of claim 1, (b) the liquid or aqueous formulation of claim 2, or (c) the vesicle, liposome, nanoparticle or nanolipid particle of claim 3, or (ii) the kit of (i) further comprising instructions for practicing the methods of any of claims 4 to 5.

72. An in vitro or ex vivo method for direct reprogramming of a first differentiated phenotype of a cell to a second differentiated phenotype, comprising:
(i) providing a differentiated cell having a first differentiated phenotype;
(ii) identifying a set of transcription factors responsible for maintaining the differentiated phenotype of the differentiated cell;
(iii) identifying a set of nucleic acid and/or protein transcription factors responsible for maintaining the second differentiated phenotype; and
(iv) simultaneously
(1) inhibiting the expression of one or more or all of the nucleic acid and/or protein transcription factors of (a)(ii), wherein by inhibiting the expression of the transcription factors the cell is unable to maintain the first differentiated phenotype; and
(2) activating the expression of the set of transcription factors of (a)(iii), wherein by activating the expression of the set of transcription factors the cell differentiates into the second differentiated phenotype,
thereby directly reprogramming the cell from a first differentiated phenotype to a second differentiated phenotype.

73. The method of claim 72, wherein the direct reprogramming step further comprises, or also comprises, contacting the cell with the composition of any of claims 1 to 24, or the liquid, gel, hydrogel, powder or aqueous formulation of claim 25, or the vesicle, liposome, nanoparticle or nanolipid particle (NLP) of claim 26.

74. The method of claim 72, wherein the expression of one or more or all of the transcription factors of (a)(ii) are by inhibited by deletion or knocking out of a gene encoding one or more of the set of transcription factors responsible for maintaining the first differentiated phenotype.

75. The method of claim 74, wherein the expression of one or more or all of the transcription factor(s) of (a)(ii) are by inhibited by deletion or inhibition of a transcript (mRNA, message) encoding one or more of a set of protein transcription factors responsible for maintaining the first differentiated phenotype, and/or the activity of one or more or all of the transcription factor(s) of (a)(ii) are inhibited by direct inhibition of the activity of one or more or all protein transcription factor(s) responsible for maintaining the first differentiated phenotype.

76. The method of claim 74, wherein the deletion or inhibition of a transcript (mRNA, message) encoding one or more of a set of protein transcription factors is by expression of or administration of a nucleic acid or protein that is inhibitory to the one or more of the set of protein transcription factors, or an antibody directly inhibits the activity of one or more or all protein transcription factor(s) responsible for maintaining the first differentiated phenotype.

77. The method of claim 76, wherein the nucleic acid that is inhibitory to the one or more of the set of protein transcription factors comprises an miRNA, an siRNA, a ribozyme and/or an antisense nucleic acid.

78. The method of claim 77, wherein one of the transcription factors inhibited is Pax5, or the method of (a)(iv)(1), further comprising inhibiting or knocking out the expression of Pax5.

79. The method of any of claims 72 to 78, wherein the method further comprises addition of a histone deacetylase inhibitor.

80. The method of claim 79, wherein the histone deacetylase inhibitor comprises a valproic acid (VPA).

81. The method of any of claims 72 to 80, further comprising expressing or upregulating a methyltransferase gene or enzyme to maintain the second differentiated phenotype.

82. The method of any of claims 72 to 81, wherein the first differentiated phenotype is a keratinocyte that is reprogrammed to a second differentiated phenotype selected from the group consisting of a nerve cell or an astrocyte.

83. The method of any of claims 72 to 82, further comprising implanting the re-programmed differentiated cell in a vessel, tissue or organ.

84. The method of claim 83, wherein the re-programmed differentiated cell is implanted in the vessel, tissue or organ ex vivo or in vivo.

85. The method of any of claims 72 to 84, further comprising implanting the re-programmed differentiated cell in an individual in need thereof.

86. A re-programmed differentiated cell made by practicing the method of any of claims 72 to 84, wherein the de-differentiated or re-programmed cell is a mammalian cell.

87. The re-programmed differentiated cell of claim 86, wherein the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

88. A nucleic acid comprising or consisting of (a) a nucleic acid sequence as set forth in SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, or SEQ ID NO:30, or (b) a nucleic acid sequence encoding an amino acid sequence as set forth in SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.

89. A polypeptide having an amino acid sequence comprising SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, or SEQ ID NO:31.


SEQUENCE LISTING (31 sequences)

SEQ ID NO: 1 - protein, 23 aa, ARTIFICIAL SEQUENCE
  SYNTHESIZED PEPTIDE
CXXXXCXXXFXXXXXLXXHXXXH

SEQ ID NO: 2 - protein, 7 aa, artificial sequence
  synthesized peptide
PKKKRKV

SEQ ID NO: 3 - protein, 4 aa, artificial sequence
  synthesized peptide
XXXX

SEQ ID NO: 4 - protein, 5 aa, artificial sequence
  synthetic polypeptide
PXXXX

SEQ ID NO: 5 - protein, 7 aa, artificial sequence
  synthetic polypeptide
PXXBBBX

SEQ ID NO: 6 - protein, 4 aa, artificial sequence
  synthetic polypeptide
XXXX

SEQ ID NO: 7 - protein, 16 aa, artificial sequence
  synthetic polypeptide
KRPAATKKAGQAKKKK

SEQ ID NO: 8 - DNA, 882 bp, artificial sequence
  synthetic polynucleotide
atgggtcgtagacgcaggcgtagacgcaggcgtggtggcggtccgaagaaaaagcgtaaagtgggcggtggcggatccacgggtgagaagccgtataaatgtcccgaatgtggtaaaagttttagccgctcgaccgatctgcaaagacatcaacgcacccataccggcgaaaaaccatacaaatgtccggagtgcggcaaatctttctcgcgcagcgataacttgcagcagcatcagagaactcacactggcgagaagccctacaagtgccccgaatgcgggaagagctttagtcgttctaccaacttacaacgccaccagaggacgcataccggtgagaagccgtataaatgtcccgaatgtggtaaaagttttagcacctcggatcatctgcaaagacatcaacgcacccataccggcgaaaaaccatacaaatgtccggagtgcggcaaatctttctcgcgtagcgatcatttgcagcgccatcagagaactcacactggcgagaagccctacaagtgccccgaatgcgggaagagctttagtacctctgatcatttacaacgtcaccagaggacgcatacgggcgagaaaccgtatggtggcggtggcagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 9 - protein, 293 aa, artificial sequence
  synthetic polypeptide
MGRRRRRRRRRGGGPKKKRKVGGGGSTGEKPYKCPECGKSFSRSTDLQRHQRTHTGEKPYKCPECGKSFSRSDNLQQHQRTHTGEKPYKCPECGKSFSRSTNLQRHQRTHTGEKPYKCPECGKSFSTSDHLQRHQRTHTGEKPYKCPECGKSFSRSDHLQRHQRTHTGEKPYKCPECGKSFSTSDHLQRHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK

SEQ ID NO: 10 - DNA, 882 bp, artificial sequence
  synthetic polynucleotide
atgggtcgtagacgcaggcgtagacgcaggcgtggtggcggtccgaagaaaaagcgtaaagtgggcggtggcggatccacgggtgagaagccgtataaatgtcccgaatgtggtaaaagttttagccgctcgacccatctgcaaagacatcaacgcacccataccggcgaaaaaccatacaaatgtccggagtgcggcaaatctttctcgcgcagcgatagtttgcagaggcatcagagaactcacactggcgagaagccctacaagtgccccgaatgcgggaagagctttagtacctctgatcatttacaacgtcaccagaggacgcataccggtgagaagccgtataaatgtcccgaatgtggtaaaagttttagcagatcggatagcctgcaagaacatcaacgcacccataccggcgaaaaaccatacaaatgtccggagtgcggcaaatctttctcgcagagctcgaacttgcagcgccatcagagaactcacactggcgagaagccctacaagtgccccgaatgcgggaagagctttagtaggtctgataacttacaaagacaccagaggacgcatacgggcgagaaaccgtatggtggcggtggcagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 11 - protein, 293 aa, artificial sequence
  synthetic polypeptide
MGRRRRRRRRRGGGPKKKRKVGGGGSTGEKPYKCPECGKSFSRSTHLQRHQRTHTGEKPYKCPECGKSFSRSDSLQRHQRTHTGEKPYKCPECGKSFSTSDHLQRHQRTHTGEKPYKCPECGKSFSRSDSLQEHQRTHTGEKPYKCPECGKSFSQSSNLQRHQRTHTGEKPYKCPECGKSFSRSDNLQRHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK

SEQ ID NO: 12 - DNA, 882 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctgaaagcaacagcctgcagcgtcaccaacggacccatactggcgaaaaaccatacaaatgtcccgagtgcggcaaatctttcagcacctctgatcatttgcagcggcatcaacgcactcacactggcgagaagccctacaagtgtccggaatgcgggaagagctttagtgaaagcgatcatctgcaacgtcaccagaggacgcataccggtgagaagccgtataaatgccccgaatgtggtaaaagtttttctcgtagcgatcatctgcagcgtcaccaacggacccatactggcgaaaaaccatacaaatgtcccgagtgcggcaaatctttcagcacctctgatgatttgcagcgtcatcaacgcactcacactggcgagaagccctacaagtgtccggaatgcgggaagagctttagtcgtagcgatcatctgcaacgtcaccagaggacgcataccggagagaaaccctatggcggaggtgggagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 13 - protein, 293 aa, artificial sequence
  synthetic polypeptide
MGRRRRRRRRRGGGPKKKRKVGGGGSTGEKPYKCPECGKSFSESNSLQRHQRTHTGEKPYKCPECGKSFSTSDHLQRHQRTHTGEKPYKCPECGKSFSESDHLQRHQRTHTGEKPYKCPECGKSFSRSDHLQRHQRTHTGEKPYKCPECGKSFSTSDDLQRHQRTHTGEKPYKCPECGKSFSRSDHLQRHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK

SEQ ID NO: 14 - DNA, 882 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctaccagcgatcatctgcagactcaccaacggacccatactggcgaaaaaccatacaaatgtcccgagtgcggcaaatctttcagcaggtctaataacttgcagcggcatcaacgcactcacactggcgagaagccctacaagtgtccggaatgcgggaagagctttagtaccagcgatcatctgcaacagcaccagaggacgcataccggtgagaagccgtataaatgccccgaatgtggtaaaagtttttctcgtagcaccagtctgcagcgtcaccaacggacccatactggcgaaaaaccatacaaatgtcccgagtgcggcaaatctttcagcaggtctgatcacttgcagacccatcaacgcactcacactggcgagaagccctacaagtgtccggaatgcgggaagagctttagtcgtagcaactccctgcaacgtcaccagaggacgcataccggagagaaaccctatggcggaggtgggagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 15 - protein, 293 aa, artificial sequence
  synthetic polypeptide
MGRRRRRRRRRGGGPKKKRKVGGGGSTGEKPYKCPECGKSFSTSDHLQTHQRTHTGEKPYKCPECGKSFSRSNNLQRHQRTHTGEKPYKCPECGKSFSTSDHLQQHQRTHTGEKPYKCPECGKSFSRSTSLQRHQRTHTGEKPYKCPECGKSFSRSDHLQTHQRTHTGEKPYKCPECGKSFSRSNSLQRHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK

SEQ ID NO: 16 - DNA, 882 bp, artificial sequence
  synthetic polynucleotide
atgggtcgtagacgcaggcgtagacgcaggcgtggtggcggtccgaagaaaaagcgtaaagtgggcggtggcggatccacgggtgagaagccgtataaatgtcccgaatgtggtaaaagttttagcgaatcgaacagcctgcaaaggcatcaacgcacccataccggcgaaaaaccatacaaatgtccggagtgcggcaaatctttctcgaccagcgatcatttgcagagacatcagagaactcacactggcgagaagccctacaagtgccccgaatgcgggaagagctttagtgaatctgatcacttacaacgccaccagaggacgcataccggtgagaagccgtataaatgtcccgaatgtggtaaaagttttagcaggtcggatcatctgcaacgtcatcaacgcacccataccggcgaaaaaccatacaaatgtccggagtgcggcaaatctttctcgaccagcgatgacttgcagagacatcagagaactcacactggcgagaagccctacaagtgccccgaatgcgggaagagctttagtagatctgatcatttacaaaggcaccagaggacgcatacgggcgagaaaccgtatggtggcggtggcagccctaggccgaccgatgtgagcctgggcgatgaactgcatttagatggcgaagatgtggcgatggcccatgcggatgccctagacgattttgacctggatatgttaggcgatggtgacagccccggtccgggttttaccccgcatgatagcgcaccgtatggtgcgctagatatggcggatttcgaatttgaacagatgtttaccgatgcgctgggtattgatgaatatggcggtgctagcgattataaagatgacgatgacaaataa

SEQ ID NO: 17 - protein, 293 aa, artificial sequence; identical to SEQ ID NO: 13
  synthetic polypeptide

SEQ ID NO: 18 - DNA, 882 bp, artificial sequence
  synthetic polynucleotide
atgggtcgtagacgcaggcgtagacgcaggcgtggtggcggtccgaagaaaaagcgtaaagtgggcggtggcggatccacgggtgagaagccgtataaatgtcccgaatgtggtaaaagttttagcacctcggatcatctgcaaacccatcaacgcacccataccggcgaaaaaccatacaaatgtccggagtgcggcaaatctttctcgcgcagcaataacttgcagagacatcagagaactcacactggcgagaagccctacaagtgccccgaatgcgggaagagctttagtacctctgaccacttacaacagcaccagaggacgcataccggtgagaagccgtataaatgtcccgaatgtggtaaaagttttagcaggtcgaccagcctgcaacgccatcaacgcacccataccggcgaaaaaccatacaaatgtccggagtgcggcaaatctttctcgcgcagcgatcatttgcagacccatcagagaactcacactggcgagaagccctacaagtgccccgaatgcgggaagagctttagtagatctaacagcttacaaaggcaccagaggacgcatacgggcgagaaaccgtatggtggcggtggcagccctaggccgaccgatgtgagcctgggcgatgaactgcatttagatggcgaagatgtggcgatggcccatgcggatgccctagacgattttgacctggatatgttaggcgatggtgacagccccggtccgggttttaccccgcatgatagcgcaccgtatggtgcgctagatatggcggatttcgaatttgaacagatgtttaccgatgcgctgggtattgatgaatatggcggtgctagcgattataaagatgacgatgacaaataa

SEQ ID NO: 19 - protein, 293 aa, artificial sequence; identical to SEQ ID NO: 15
  synthetic polypeptide

SEQ ID NO: 20 - DNA, 827 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctgaaagcaacagcctgcagcgtcaccaacggacccatactggcgaaaaaccatacaaatgtcccgagtgcggcaaatctttcagcacctctgatcatttgcagcggcatcaacgcactcacactggcgagaagccctacaagtgtccggaatgcgggaagagctttagtgaaagcgatcatctgcaacgtcaccagaggacgcataccggtgagaagccgtataaatgccccgaatgtggtaaaagtttttctcgtagcgatcatctgcagcgtcaccaacggacccatactggcgaaaaaccatacaaatgtcccgagtgcggcaaatctttcagcacctctgatgatttgcagcgtcatcaacgcactcacactggcgagaagccctacaagtgtccggaatgcgggaagagctttagtcgtagcgatcatctgcaacgtcaccagaggacgcataccggagagaaaccctatggcggaggtgggagccctaggtttgatactgacctgttcgataccgacttgtttgatactgacctttttgatactgacctgttcgataccgacttgtttgatactgacctttttgatactgacctgttcgataccgacttgtttgatactgacctttttgatactgacctgttcgataccgacttggtggcggtgctagcgattataaagatgacgatgacaaataa

SEQ ID NO: 21 - protein, 275 aa, artificial sequence
  synthetic polypeptide
MGRRRRRRRRRGGGPKKKRKVGGGGSTGEKPYKCPECGKSFSESNSLQRHQRTHTGEKPYKCPECGKSFSTSDHLQRHQRTHTGEKPYKCPECGKSFSESDHLQRHQRTHTGEKPYKCPECGKSFSRSDHLQRHQRTHTGEKPYKCPECGKSFSTSDDLQRHQRTHTGEKPYKCPECGKSFSRSDHLQRHQRTHTGEKPYGGGGSPRFDTDLFDTDLFDTDLFDTDLFDTDLFDTDLFDTDLFDTDLFDTDLFDTDLFDTDLGGGASDYKDDDDK

SEQ ID NO: 22 - DNA, 827 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctaccagcgatcatctgcagactcaccaacggacccatactggcgaaaaaccatacaaatgtcccgagtgcggcaaatctttcagcaggtctaataacttgcagcggcatcaacgcactcacactggcgagaagccctacaagtgtccggaatgcgggaagagctttagtaccagcgatcatctgcaacagcaccagaggacgcataccggtgagaagccgtataaatgccccgaatgtggtaaaagtttttctcgtagcaccagtctgcagcgtcaccaacggacccatactggcgaaaaaccatacaaatgtcccgagtgcggcaaatctttcagcaggtctgatcacttgcagacccatcaacgcactcacactggcgagaagccctacaagtgtccggaatgcgggaagagctttagtcgtagcaactccctgcaacgtcaccagaggacgcataccggagagaaaccctatggcggaggtgggagccctaggtttgatactgacctgttcgataccgacttgtttgatactgacctttttgatactgacctgttcgataccgacttgtttgatactgacctttttgatactgacctgttcgataccgacttgtttgatactgacctttttgatactgacctgttcgataccgacttggtggcggtgctagcgattataaagatgacgatgacaaataa

SEQ ID NO: 23 - protein, 275 aa, artificial sequence
  synthetic polypeptide
MGRRRRRRRRRGGGPKKKRKVGGGGSTGEKPYKCPECGKSFSTSDHLQTHQRTHTGEKPYKCPECGKSFSRSNNLQRHQRTHTGEKPYKCPECGKSFSTSDHLQQHQRTHTGEKPYKCPECGKSFSRSTSLQRHQRTHTGEKPYKCPECGKSFSRSDHLQTHQRTHTGEKPYKCPECGKSFSRSNSLQRHQRTHTGEKPYGGGGSPRFDTDLFDTDLFDTDLFDTDLFDTDLFDTDLFDTDLFDTDLFDTDLFDTDLFDTDLGGGASDYKDDDDK

SEQ ID NO: 24 - DNA, 852 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctgaaagcaacagcctgcagcgtcaccaacggacccatactggcgaaaaaccatacaaatgtcccgagtgcggcaaatctttcagcacctctgatcatttgcagcggcatcaacgcactcacactggcgagaagccctacaagtgtccggaatgcgggaagagctttagtgaaagcgatcatctgcaacgtcaccagaggacgcataccggtgagaagccgtataaatgccccgaatgtggtaaaagtttttctcgtagcgatcatctgcagcgtcaccaacggacccatactggcgaaaaaccatacaaatgtcccgagtgcggcaaatctttcagcacctctgatgatttgcagcgtcatcaacgcactcacactggcgagaagccctacaagtgtccggaatgcgggaagagctttagtcgtagcgatcatctgcaacgtcaccagaggacgcataccggagagaaaccctatggcggaggtgggagccctaggaccctggtgacctttaaggatgtgttcgtcgattttaccagggaggaatggaaactgttggacaccgcccaacagattgtctatagaaatgtgatgctggagaactataagaatctggtgagcttgggctatcagctgaccaaacccgacgtgattctgagactcgaaaagggcgaggaaccctggctggtgggcggagctagcgattataaagatgacgatgacaaataa

SEQ ID NO: 25 - protein, 283 aa, artificial sequence
  synthetic polypeptide
MGRRRRRRRRRGGGPKKKRKVGGGGSTGEKPYKCPECGKSFSESNSLQRHQRTHTGEKPYKCPECGKSFSTSDHLQRHQRTHTGEKPYKCPECGKSFSESDHLQRHQRTHTGEKPYKCPECGKSFSRSDHLQRHQRTHTGEKPYKCPECGKSFSTSDDLQRHQRTHTGEKPYKCPECGKSFSRSDHLQRHQRTHTGEKPYGGGGSPRTLVTFKDVFVDFTREEWKLLDTAQQIVYRNVMLENYKNLVSLGYQLTKPDVILRLEKGEEPWLVGGASDYKDDDDK

SEQ ID NO: 26 - DNA, 852 bp, artificial sequence
  synthetic polynucleotide
atgggtcggagacgcaggcggagacgcaggcgtggtggcgggcccaaaaagaaacggaaagtgggcggtgggggatccacgggggagaagccgtataaatgccccgaatgtggtaaaagtttttctaccagcgatcatctgcagactcaccaacggacccatactggcgaaaaaccatacaaatgtcccgagtgcggcaaatctttcagcaggtctaataacttgcagcggcatcaacgcactcacactggcgagaagccctacaagtgtccggaatgcgggaagagctttagtaccagcgatcatctgcaacagcaccagaggacgcataccggtgagaagccgtataaatgccccgaatgtggtaaaagtttttctcgtagcaccagtctgcagcgtcaccaacggacccatactggcgaaaaaccatacaaatgtcccgagtgcggcaaatctttcagcaggtctgatcacttgcagacccatcaacgcactcacactggcgagaagccctacaagtgtccggaatgcgggaagagctttagtcgtagcaactccctgcaacgtcaccagaggacgcataccggagagaaaccctatggcggaggtgggagccctaggaccctggtgacctttaaggatgtgttcgtcgattttaccagggaggaatggaaactgttggacaccgcccaacagattgtctatagaaatgtgatgctggagaactataagaatctggtgagcttgggctatcagctgaccaaacccgacgtgattctgagactcgaaaagggcgaggaaccctggctggtgggcggagctagcgattataaagatgacgatgacaaataa

SEQ ID NO: 27 - protein, 283 aa, artificial sequence
  synthetic polypeptide
MGRRRRRRRRRGGGPKKKRKVGGGGSTGEKPYKCPECGKSFSTSDHLQTHQRTHTGEKPYKCPECGKSFSRSNNLQRHQRTHTGEKPYKCPECGKSFSTSDHLQQHQRTHTGEKPYKCPECGKSFSRSTSLQRHQRTHTGEKPYKCPECGKSFSRSDHLQTHQRTHTGEKPYKCPECGKSFSRSNSLQRHQRTHTGEKPYGGGGSPRTLVTFKDVFVDFTREEWKLLDTAQQIVYRNVMLENYKNLVSLGYQLTKPDVILRLEKGEEPWLVGGASDYKDDDDK

SEQ ID NO: 28 - DNA, 882 bp, artificial sequence
  synthetic polynucleotide
atgggtcggagacgcaggcggagacgcaggcgtggtggcgggcccaaaaagaaacggaaagtgggcggtgggggatccacgggtgagaagccgtataaatgtcccgaatgtggtaaaagttttagcaggtcgacccatctgcaacagcatcaacgcacccataccggcgaaaaaccatacaaatgtccggagtgcggcaaatctttctcgacgagcacccacttgcagaggcatcagagaactcacactggcgagaagccctacaagtgccccgaatgcgggaagagctttagtcgttctgataatttacaaacccaccagaggacgcataccggtgagaagccgtataaatgtcccgaatgtggtaaaagttttagccgttcgaatcatctgcaaagacatcaacgcacccataccggcgaaaaaccatacaaatgtccggagtgcggcaaatctttctcgaccagcgaccacttgcagagacatcagagaactcacactggcgagaagccctacaagtgccccgaatgcgggaagagctttagtagatctgatcatttacaacgtcaccagaggacgcatacgggcgagaaaccgtatggtggcggtggcagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 29 - protein, 293 aa, artificial sequence
  synthetic polypeptide
MGRRRRRRRRRGGGPKKKRKVGGGGSTGEKPYKCPECGKSFSRSTHLQQHQRTHTGEKPYKCPECGKSFSTSTHLQRHQRTHTGEKPYKCPECGKSFSRSDNLQTHQRTHTGEKPYKCPECGKSFSRSNHLQRHQRTHTGEKPYKCPECGKSFSTSDHLQRHQRTHTGEKPYKCPECGKSFSRSDHLQRHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK

SEQ ID NO: 30 - DNA, 882 bp, artificial sequence
  synthetic polynucleotide
atgggtcggagacgcaggcggagacgcaggcgtggtggcgggcccaaaaagaaacggaaagtgggcggtgggggatccacgggtgagaagccgtataaatgtcccgaatgtggtaaaagttttagcagatcgaattctctgcaacagcatcaacgcacccataccggcgaaaaaccatacaaatgtccggagtgcggcaaatctttctcgagaagcacccacttgcagaggcatcagagaactcacactggcgagaagccctacaagtgccccgaatgcgggaagagctttagtaggtctgatcacttacaaaggcaccagaggacgcataccggtgagaagccgtataaatgtcccgaatgtggtaaaagttttagccgttcggaccacctgcaaagacatcaacgcacccataccggcgaaaaaccatacaaatgtccggagtgcggcaaatctttctcgcgtagcgatcatttgcagacgcatcagagaactcacactggcgagaagccctacaagtgccccgaatgcgggaagagctttagtacctctaactctttacaaacgcaccagaggacgcatacgggcgagaaaccgtatggtggcggtggcagccctaggcccaccgatgtgagcctgggcgatgaactccacctggatggcgaagacgttgctatggcccatgcagatgctcttgatgactttgatctggacatgcttggagatggtgacagccctggccccggatttacccctcatgatagcgcaccctatggagcactggatatggccgatttcgaatttgaacagatgtttaccgatgcactcggaattgatgaatatggcggagctagcgattataaagatgacgatgacaaataa

SEQ ID NO: 31 - protein, 293 aa, artificial sequence
  synthetic polypeptide
MGRRRRRRRRRGGGPKKKRKVGGGGSTGEKPYKCPECGKSFSRSNSLQQHQRTHTGEKPYKCPECGKSFSRSTHLQRHQRTHTGEKPYKCPECGKSFSRSDHLQRHQRTHTGEKPYKCPECGKSFSRSDHLQRHQRTHTGEKPYKCPECGKSFSRSDHLQTHQRTHTGEKPYKCPECGKSFSTSNSLQTHQRTHTGEKPYGGGGSPRPTDVSLGDELHLDGEDVAMAHADALDDFDLDMLGDGDSPGPGFTPHDSAPYGALDMADFEFEQMFTDALGIDEYGGASDYKDDDDK